(19) United States
(12) Patent Application Publication
(10) Pub. No.: US 2012/0264753 A1
(43) Pub. Date: Oct. 18, 2012

(54) NOVEL 1-(BIPHENYL-4-YL-METHYL)-1H-IMIDAZOLE DERIVATIVE AND PHARMACEUTICAL PRODUCT CONTAINING SAME

(75) Inventors: Toru Miura, Tokyo (JP); Hajime Yamada, Tokyo (JP); Junya Tagashira, Tokyo (JP); Gen Watanabe, Tokyo (JP); Ryohei Sekimoto, Tokyo (JP); Rie Ishida, Tokyo (JP); Hitomi Aoki, Tokyo (JP); Tadaaki Ohgiya, Tokyo (JP)

(73) Assignee: KOWA COMPANY, LTD., Nagoya-shi, Aichi (JP)

(21) Appl. No.: 13/511,876

(22) PCT Filed: Dec. 22, 2010
(86) PCT No.: PCT/JP2010/007418

§ 371 Date: May 24, 2012

(30) Foreign Application Priority Data
Dec. 22, 2009 (JP) 2009-291379

(51) Int. Cl. A61K 31/4178; A61K 31/4245; C07D 413/14; A61K 31/506; C07D 403/14; A61K 31/5377; C07D 403/10; A61P 9/00; A61P 9/12; A61P 9/10; A61P 13/12; A61P 3/00; A61P 3/10; A61P 25/00; C07D 413/10

(52) U.S. Cl. 514/236.2; 548/132; 514/364; 544/298; 514/269; 544/138; 548/253; 514/381; 544/139

(57) ABSTRACT

Disclosed is a novel 1-(biphenyl-4-yl-methyl)-1H-imidazole derivative represented by general formula (I), which has both angiotensin II receptor antagonist activity and PPAR-γ activating activity and is useful as a prophylactic and/or therapeutic agent for cardiovascular diseases and metabolic diseases. Also disclosed is a pharmaceutical composition which contains the novel 1-(biphenyl-4-yl-methyl)-1H-imidazole derivative. In general formula (I), ring A represents a group represented by formula (II) or (III); R¹ represents a C₁₋₆ alkyl group; R² represents a C₁₋₆ alkyl group which may be substituted by a hydroxy group, or a group represented by formula —CO—R⁴ (wherein R⁴ represents a hydroxy group, a C₁₋₆ alkoxy group, an amino group, a mono(C₁₋₆ alkyl)amino group, a di(C₁₋₆ alkyl)amino group, a morpholino group, a piperidino group or a pyrrolidino group); R³ represents a halogen atom or a C₁₋₆ alkoxy group; and X and Y may be the same or different and each represents a nitrogen atom or CH.

### TECHNICAL FIELD

The present invention relates to a novel 1-(biphenyl-4-yl-methyl)-1H-imidazole derivative that has both angiotensin II antagonistic activity and PPARγ activation activity, and a pharmaceutical agent containing the same.

### BACKGROUND ART

In recent years, disorders like diabetes, hypertension, dyslipidemia and obesity which can be a risk factor for arteriosclerotic disorders have been rapidly increasing due to changes in life style with improvements in living standard, i.e., high calorie and high cholesterol type diet, obesity, lack of exercise, aging, and the like. It is known that; although being a risk factor independent of each other, overlap of the disorders can cause an occurrence of arteriosclerotic disorders at higher frequency or aggravation of the disorders. As such, with the understanding of a condition having a plurality of risk factors for arteriosclerotic disorders as metabolic syndrome, efforts have been made to elucidate the cause of the syndrome and to develop a therapeutic method therefor.

Angiotensin II (herein below, it may be also abbreviated as AII) is a peptide that is found to be an intrinsic pressor substance produced by renin-angiotensin system (i.e., RA system). It is believed that pharmacological inhibition of angiotensin II activity can lead to treatment or prevention of circulatory disorders like hypertension. Accordingly, an inhibitor for angiotensin converting enzyme (ACE) which inhibits the enzyme promoting the conversion of angiotensin I (AI) to angiotensin II has been clinically used as an inhibitory agent for RA system. Furthermore, an orally administrable AII receptor blocker (Angiotensin Receptor Blocker: ARB) has been developed, and losartan, candesartan, telmisartan, valsartan, olmesartan, and irbesartan, and the like are already clinically used as a hypotensive agent. It is reported by many clinical or basic studies that, as having not only a hypotensive activity but also other various activities including an anti-inflammatory activity, an endothelial function improving activity, a cardiovascular remodeling inhibiting activity, an oxidation stress inhibiting activity, a proliferation factor inhibiting activity, and insulin resistance improving activity, and the like, ARB is useful for cardiovascular disorders, renal diseases, and arteriosclerosis, and the like (Non-Patent Documents 1 and 2). Most recently, it is also reported that ARB particularly has a kidney protecting activity which does not depend on a hypotensive activity (Non-Patent Document 3).

Meanwhile, three isoforms, i.e., α, γ, and δ, have been identified so far as peroxisome proliferator-activated receptors (PPARs) which belong to a nuclear receptor superfamily. Among them, PPARγ is an isoform that is most abundantly expressed in an adipose tissue and it plays an important role in differentiation of adipocytes or metabolism of glycolipids. Currently, thiazolidinedione derivatives (i.e., TZD) like pioglitazone or rosiglitazone are clinically used as a therapeutic agent for diabetes having PPARγ activation activity, and they are known to have an activity of improving insulin resistance, glucose tolerance, and lipid metabolism, and the like. Further, it is recently reported that, based on activation of PPARγ, TZD exhibits various activities including a hypotensive activity, an anti-inflammatory activity, an endothelial function improving activity, a proliferation factor inhibiting activity, and an activity of interfering RA system, and the like. It is also reported that, according to such multiple activities, TZD shows a kidney protecting activity particularly in diabetic nephropathy without depending on blood sugar control (Non-Patent Documents 4, 5, 6, 7, and 8). Meanwhile, there is also a concern regarding adverse effects of TZD caused by PPARγ activation like body fluid accumulation, body weight gain, peripheral edema, and pulmonary edema (Non-Patent Documents 9 and 10).

It has been recently reported that telmisartan has a PPARγ activation activity (Non-Patent Document 11). It has been also reported that the irbesartan has the same activity (Non-Patent Document 12). These compounds have both a RA system inhibiting activity and a PPARγ activation activity, and thus are expected to be used as an integrated agent for prevention and/or treatment of circulatory disorders (e.g., hypertension, heart diseases, angina pectoris, cerebrovascular disorders, cerebral circulatory disorders, ischemic peripheral circulatory disorders, and renal diseases, and the like) or diabetes-related disorders (e.g., Type II diabetes, diabetic complications, insulin resistant syndrome, metabolic syndrome, hyperinsulinemia, and the like) without increasing a risk of body fluid accumulation, body weight gain, peripheral edema, pulmonary edema, or congestive heart failure that are concerned over the use of MD (Patent Document 1). Among them, for diabetic nephropathy, a synergistic prophylactic and/or therapeutic effect is expected from multiple kidney protecting activity based on activities of RA system inhibition and PPARγ activation.

As compounds having such activities, pyrimidine and triazine derivatives (Patent Document 1), imidazopyridine derivatives (Patent Document 2), indole derivatives (Patent Document 3), imidazole derivatives (Patent Document 4), and condensed ring derivatives (Patent Document 5) have been reported. However, there is no description or suggestion related to the 1-(biphenyl-4-yl-methyl)-1H-imidazole derivatives in which an aromatic compound is substituted at position 4.

Meanwhile, a compound represented by the following formula:

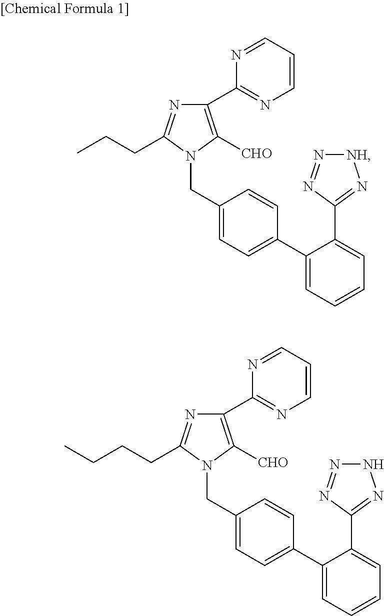

is described in the Example 41 and the Example 62 of Patent Document 6. Further, a compound represented by the following formula:

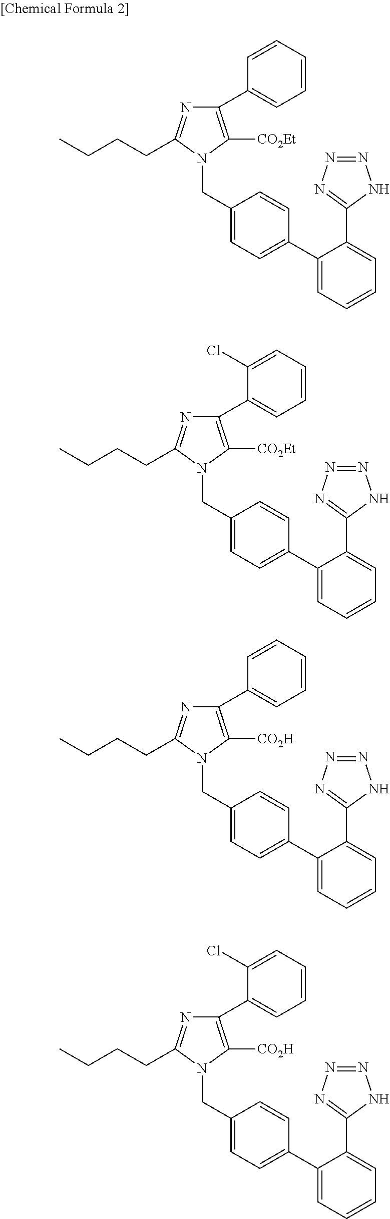

is disclosed in Non-Patent Document 13. In this regard, although in Non-Patent Document 13 or Patent Document 6, there are descriptions that the disclosed compounds exhibit an AII receptor antagonistic activity and have a therapeutic effect on high blood pressure, there is no description or suggestion that the compounds of the document has a PPARγ activation activity or a therapeutic effect on diabetes, obesity, or metabolic syndrome.

### PRIOR ART LITERATURE

### Patent Document

Patent Document 1: WO 2008/062905
Patent Document 2: WO 2008/084303
Patent Document 3: WO 2008/096820
Patent Document 4: WO 2008/096829
Patent Document 5: WO 2008/143262
Patent Document 6: WO 1991/00227

### Non-Patent Document

Non-Patent Document 1: AMER. J. Hypertension, 18, 720 (2005)
Non-Patent Document 2: Current Hypertension Report, 10, 261 (2008)
Non-Patent Document 3: Diabetes Care, 30, 1581 (2007)
Non-Patent Document 4: Kidney Int., 70, 1223 (2006)
Non-Patent Document 5: Circulation, 108, 2941 (2003)
Non-Patent Document 6: Best Pract. Res. Clin. Endocrinol. Metab., 21 (4), 687 (2007)
Non-Patent Document 7: Diab. Vasc. Dis. Res., 1 (2), 76 (2004)
Non-Patent Document 8: Diab. Vasc. Dis. Res., 2 (2), 61 (2005)
Non-Patent Document 9: J. Clin. Invest., 116 (3), 581 (2006)
Non-Patent Document 10: FASEB J., 20 (8), 1203 (2006)
Non-Patent Document 11: Hypertension, 43, 993 (2004)
Non-Patent Document 12: Circulation, 109, 2054 (2004)
Non-Patent Document 13: J. Med. Chem., 36, 3595-3605 (1993)

### SUMMARY OF THE INVENTION

### Problems to be Solved by the Invention

An object of the present invention is to provide a novel compound that is useful as a pharmaceutical agent for preventing and/or treating hypertension as a circulatory disorder, diabetes as a metabolic disease, or the like, and a pharmaceutical composition using the same.

### Means for Solving the Problems

As a result of intensive studies to achieve the purpose described above, the inventors of the invention found that the compound represented by the formula (I) below is a compound that has excellent angiotensin II antagonistic activity and PPARγ activation activity, and therefore completed the invention.

Specifically, the present invention relates to the following inventions.

[1] A 1-(biphenyl-4-yl-methyl)-1H-imidazole derivative represented by the formula (I) below or a salt thereof, or a solvate thereof:

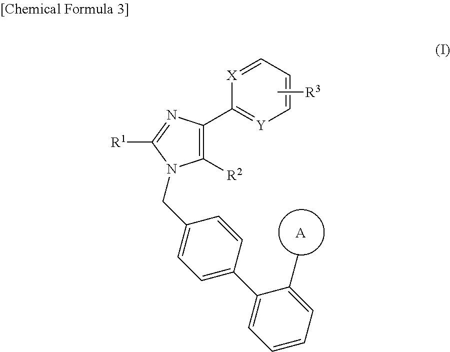

[in the formula, ring A represents the following formula (II) or the following formula (III):

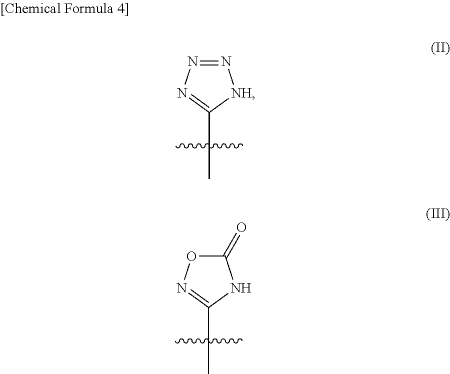

R¹ represents a C₁₋₆ alkyl group,

R² represents a C₁₋₆ alkyl group which may be substituted with a hydroxyl group, or —CO—R⁴ (wherein, R⁴ represents a hydroxyl group, a C₁₋₆ alkoxy group, an amino group, a mono(C₁₋₆ alkyl)amino group, a di(C₁₋₆ alkyl)amino group, a morpholino group, a piperidino group, or a pyrrolidino group),

R³ represents a halogen atom or a C₁₋₆ alkoxy group, and

X and Y, which are the same or different each other, represent a nitrogen atom or CH].

[2] The compound described in [1] above, or the salt thereof, or the solvate thereof, in which the compound represented by the formula (I) is at least one compound selected from a group consisting of:
3-[4′-[{2-butyl-4-(4-fluorophenyl)-5-(hydroxymethyl)-1H-imidazol-1-yl}methyl]biphenyl-2-yl]-1,2,4-oxadiazol-5(4H)-one,
methyl 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid,
2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid,
3-[4′-[{2-butyl-4-(5-ethoxypyrimidin-2-yl)-5-(hydroxy methyl)-1H-imidazol-1-yl}methyl]-biphenyl-2-yl]-1,2,4-oxadiazol-5(4H)-one,
[1-[{2′-(1H-tetrazol-5-yl)biphenyl-4-yl}methyl]-2-butyl-4-(5-ethoxypyrimidin-2-yl)-1H-imidazol-5-yl]methanol,
methyl 2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid,
2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid,
methyl 1-[{2′-(1H-tetrazol-5-yl)biphenyl-4-yl}methyl]-2-butyl-4-(5-ethoxypyrimidin-2-yl)-1H-imidazole-5-carboxylic acid,
1-[{2′-(1H-tetrazol-5-yl)biphenyl-4-yl}methyl]-2-butyl-4-(5-ethoxypyrimidin-2-yl)-1H-imidazole-5-carboxylic acid,
2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
2-butyl-N-ethyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
2-butyl-N,N-diethyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
3-[4′-[{2-butyl-4-(4-fluorophenyl)-5-(morpholin-4-carbonyl)-1H-imidazol-1-yl}methyl]biphenyl-2-yl]-1,2,4-oxadiazol-5(4H)-one,
3-[4′-[{2-butyl-4-(4-fluorophenyl)-5-(pyrrolidin-1-carbonyl)-1H-imidazol-1-yl}methyl]biphenyl-2-yl]-1,2,4-oxadiazol-5(4H)-one,
1-[{2′-(1H-tetrazol-5-yl)biphenyl-4-yl}methyl]-2-butyl-4-(4-fluorophenyl)-1H-imidazole-5-carboxamide,
1-[{2′-(1H-tetrazol-5-yl)biphenyl-4-yl}methyl]-2-butyl-N-ethyl-4-(4-fluorophenyl)-1H-imidazole-5-carboxamide,
1-[{2′-(1H-tetrazol-5-yl)biphenyl-4-yl}methyl]-2-butyl-N,N-diethyl-4-(4-fluorophenyl)-1H-imidazole-5-carboxamide
[1-[{2′-(1H-tetrazol-5-yl)biphenyl-4-yl}methyl]-2-butyl-4-(4-fluorophenyl)-1H-imidazol-5-yl](morpholino)methanone, and
[1-[{2′-(1H-tetrazol-5-yl)biphenyl-4-yl}methyl]-2-butyl-4-(4-fluorophenyl)-1H-imidazol-5-yl](pyrrolidin-1-yl)methanone.

The alkyl group such as butyl in the nomenclature of the above-mentioned compounds represents a straight (normal) chain unless particularly designated.

[3] A pharmaceutical composition containing the compound described in [1] or [2] above, or the salt thereof, or the solvate thereof, and a pharmaceutically acceptable carrier.

[4] A pharmaceutical composition which has both angiotensin II receptor antagonistic activity and PPARγ activation activity and contains as an effective component the compound or the salt thereof, or the solvate thereof described in [1] or [2] above.

[5] An agent for preventing and/or treating a circulatory disorder which contains as an effective component the compound or the salt thereof, or the solvate thereof described in [1] or [2] above.

[6] The agent for preventing and/or treating a circulatory disease described in [5] above, in which the circulatory disease is hypertension, heart disease, angina pectoris, cerebral vascular accident, cerebrovascular disorder, ischemic peripheral circulatory disorder, kidney disease, or arteriosclerosis.

[7] An agent for preventing and/or treating a metabolic disease which contains as an effective component the compound or the salt thereof, or the solvate thereof described in [1] or [2] above.

[8] The agent for preventing and/or treating a metabolic disease described in [7] above, in which the metabolic disease is Type II diabetes, diabetic complication (diabetic retinopathy, diabetic neuropathy, or diabetic nephropathy), insulin resistant syndrome, metabolic syndrome, or hyperinsulinemia.

[9] A method of preventing and/or treating a circulatory disease containing administering an effective amount of the compound or the salt thereof, or the solvate thereof described in [1] or [2] above to a patient in need of the treatment.

[10] A method of preventing and/or treating a metabolic disease containing administering an effective amount of the compound or the salt thereof, or the solvate thereof described in [1] or [2] above to a patient in need of the treatment.

[11] Use of the compound or the salt thereof, or the solvate thereof described in [1] or [2] above for production of a preparation used for prevention and/or treatment of a circulatory disease.

[12] Use of the compound or the salt thereof, or the solvate thereof described in [1] or [2] above for producing a preparation for preventing and/or treating a metabolic disease.

[13] The compound or the salt thereof, or the solvate thereof described in [1] or [2] above as an agent for prevention and/or treatment having both an angiotensin II receptor antagonist activity and a PPARγ activation activity.

### Effects of the Invention

The 1-(biphenyl-4-yl-methyl)-1H-imidazole derivative represented by the formula (I) of the invention or a salt thereof, or a solvate thereof exhibits a potent antagonistic activity for an angiotensin II receptor, and can be appropriately used as an effective component for an agent for preventing and/or treating a disease related with angiotensin II, for example a circulatory disease such as hypertension, heart disease, angina pectoris, cerebral vascular accident, cerebrovascular disorder, ischemic peripheral circulatory disorder, kidney disease, and arteriosclerosis.

Further, the 1-(biphenyl-4-yl-methyl)-1H-imidazole derivative represented by the formula (I) of the invention or a salt thereof, or a solvate thereof has a PPARγ activation activity and can be appropriately used as an effective component for an agent for preventing and/or treating a disease related with PPARγ, for example metabolic disease such as arteriosclerosis, Type II diabetes mellitus, diabetic complication (diabetic retinopathy, diabetic neuropathy, or diabetic nephropathy), insulin resistance syndrome, syndrome X, metabolic syndrome, and hyperinsulinemia.

Still further, the 1-(biphenyl-4-yl-methyl)-1H-imidazole derivative represented by the formula (I) of the invention, or a salt thereof, or a solvate thereof has both an antagonistic activity for an angiotensin II receptor and PPARγ activation activity and can be appropriately used as an effective component for an agent for preventing and/or treating a disease related with both angiotensin II and PPARγ, for example, arteriosclerosis, diabetic nephropathy, insulin resistance syndrome, syndrome X, and metabolic syndrome.

### MODES FOR CARRYING OUT THE INVENTION

The “halogen atom” as used herein includes a fluorine atom, a chlorine atom, a bromine atom, an iodine atom, and the like.

The “C₁₋₆ alkyl group” and the “C₁₋₆ alkyl” as used herein mean a linear or a branched hydrocarbon group having 1 to 6 carbon atoms, and examples thereof include a methyl group, an ethyl group, a n-propyl group, an isopropyl group, a n-butyl group, an isobutyl group, a tert-butyl group, a n-pentyl group, a 2-methylbutyl group, a 2,2-dimethylpropyl group, a n-hexyl group, and the like. Further, the “C₁₋₆ alkyl group which may be substituted with a hydroxyl group” means a “C₁₋₆ alkyl group” to which 1 to 3, and preferably 1 hydroxyl group is bonded, and examples thereof include a hydroxymethyl group, a 1-hydroxyethyl group, a 2-hydroxyethyl group, a 1-hydroxypropyl group, a 2-hydroxypropyl group, a 3-hydroxypropyl group, a 4-hydroxybutyl group, and the like.

The “C₁₋₆ alkoxy group” as used herein means a linear or a branched alkoxy group having 1 to 6 carbon atoms, and examples thereof include a methoxy group, an ethoxy group, a propoxy group, an isopropoxy group, a butoxy group, an isobutoxy group, a sec-butoxy group, a tert-butoxy group, a pentoxy group, an isopentoxy group, a neopentoxy group, a hexyloxy group, an isohexyloxy group, and the like.

The “mono alkylamino group” as used herein means an amino group having one alkyl group bonded to the nitrogen atom of the amino group. Thus, examples of the “mono(C₁₋₆ alkyl)amino group” include a methylamino group, an ethylamino group, a propylamino group, an isopropylamino group, a butylamino group, a sec-butylamino group, a tert-butylamino group, a pentylamino group, an isopentylamino group, a neopentylamino group, a 1-methylbutylamino group, a 1-ethylpropylamino group, a hexylamino group, an isohexylamino group, a 4-methylpentylamino group, a 3-methylpentylamino group, a 2-methylpentylamino group, a 1-methylpentylamino group, a 3,3-dimethylbutylamino group, a 2,2-dimethylbutylamino group, a 1,1-dimethylbutylamino group, a 1,2-dimethylbutylamino group, a 1,3-dimethylbutylamino group, a 2,3-dimethylbutylamino group, a 1-ethylbutylamino group, a 2-ethylbutylamino group, and the like.

The “dialkylamino group” as used herein means an amino group having two alkyl groups which may be the same or different each other, bonded to the nitrogen atom of the amino group. Thus, examples of the “di(C₁₋₆ alkyl)amino group” include a dimethylamino group, a methylethyl amino group, a diethylamino group, a methylpropylamino group, an ethylpropylamino group, a dipropylamino group, a methylisopropylamino group, an ethyl isopropylamino group, a diisopropylamino group, a methylbutylamino group, an ethylbutylamino group, a propylbutylamino group, a dibutylamino group, a di-sec-butylamino group, a di-tert-butylamino group, a dipentylamino group, a dihexylamino group, and the like.

Examples of the preferred mode of the invention include the followings.

In the formula (I), the C₁₋₆ alkyl group as R¹ is preferably a C₁₋₄ alkyl group, more preferably a C₂₋₄ alkyl group, and still more preferably an n-butyl group.

In the formula (I), the C₁₋₆ alkyl group which may be substituted with a hydroxyl group as R² is preferably a C₁₋₄ alkyl group which may be substituted with a hydroxyl group, and more preferably a hydroxymethyl group.

In the formula (I), the halogen atom as R³ is preferably a fluorine atom.

In the formula (I), the C₁₋₆ alkoxy group as R³ is preferably a C₁₋₄ alkoxy group, and more preferably an ethoxy group.

In the formula (I), the substitution position of R³ can be any one of the meta position and para position relative to the binding position of an imidazole ring. However, the para position is more preferable.

In the formula (I), the C₁₋₆ alkoxy group as R³ is preferably a C₁₋₄ alkoxy group, and more preferably a methoxy group.

In the formula (I), the mono(C₁₋₆ alkyl)amino group as R⁴ is preferably a mono(C₁₋₄ alkyl)amino group, and more preferably an ethylamino group.

In the formula (I), the di(C₁₋₆ alkyl)amino group as R⁴ is preferably a di(C₁₋₄ alkyl)amino group, and more preferably a diethylamino group.

In the formula (I), it is more preferable that R⁴ is either a morpholino group or a pyrrolidino group.

The ring A in the formula (I) is preferably an oxadiazole ring of the formula (III) when the focus is made on the PPARγ activation activity. When the focus is made on the angiotensin II receptor antagonistic activity, the tetrazole ring of the formula (II) is preferable.

Further, when X and Y are CH, R² is preferably an amide derivative to have a —CON group. When X and Y are N, R² is preferably a C₁₋₄ alkyl group which may be substituted with a hydroxy group. When X and Y are N, R² is particularly preferably a hydroxymethyl group.

More preferred examples of the 1-(biphenyl-4-yl-methyl)-1H-imidazole derivative represented by the formula (I) include at least one compound that is selected from the group consisting of following compounds:
3-[4′-[{2-butyl-4-(4-fluorophenyl)-5-(hydroxymethyl)-1H-imidazol-1-yl}methyl]biphenyl-2-yl]-1,2,4-oxadiazol-5(4H)-one,
methyl 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid,
2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid,
3-[4′-[{2-butyl-4-(5-ethoxypyrimidin-2-yl)-5-(hydroxy methyl)-1H-imidazol-1-yl}methyl]-biphenyl-2-yl]-1,2,4-oxadiazol-5(4H)-one,
[1-[{2′-(1H-tetrazol-5-yl)biphenyl-4-yl}methyl]-2-butyl-4-(5-ethoxypyrimidin-2-yl)-1H-imidazol-5-yl]methanol, methyl
2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid,
2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid,
methyl 1-[{2′-(1H-tetrazol-5-yl)biphenyl-4-yl}methyl]-2-butyl-4-(5-ethoxypyrimidin-2-yl)-1H-imidazole-5-carboxylic acid,
1-[{2′-(1H-tetrazol-5-yl)biphenyl-4-yl}methyl]-2-butyl-4-(5-ethoxypyrimidin-2-yl)-1H-imidazole-5-carboxylic acid,
2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
2-butyl-N-ethyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
2-butyl-N,N-diethyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
3-[4′-[{2-butyl-4-(4-fluorophenyl)-5-(morpholin-4-car bonyl)-1H-imidazol-1-yl}methyl]biphenyl-2-yl]-1,2,4-oxadiazol-5(4H)-one,
3-[4′-[{2-butyl-4-(4-fluorophenyl)-5-(pyrrolidin-1-carbonyl)-1H-imidazol-1-yl}methyl]biphenyl-2-yl]-1,2,4-oxadiazol-5(4H)-one,
1-[{2′-(1H-tetrazol-5-yl)biphenyl-4-yl}methyl]-2-butyl-4-(4-fluorophenyl)-1H-imidazole-5-carboxamide,
1-[{2′-(1H-tetrazol-5-yl)biphenyl-4-yl}methyl]-2-butyl-N-ethyl-4-(4-fluorophenyl)-1H-imidazole-5-carboxamide,
1-[{2′-(1H-tetrazol-5-yl)biphenyl-4-yl}methyl]-2-butyl-N,N-diethyl-4-(4-fluorophenyl)-1H-imidazole-5-carboxamide
(1-([2′-(1H-tetrazol-5-yl)biphenyl-4-yl)methyl]-2-butyl-4-(4-fluorophenyl)-1H-imidazol-5-yl)(morpholino)methanone, and
[1-[{2′-(1H-tetrazol-5-yl)biphenyl-4-yl}methyl]-2-butyl-4-(4-fluorophenyl)-1H-imidazol-5-yl](pyrrolidin-1-yl)methanone.

Still more preferred examples of the 1-(biphenyl-4-yl-methyl)-1H-imidazole derivative represented by the formula (I) include at least one compound that is selected from the group consisting of following compounds:
methyl 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid,
3-[4′-[{2-butyl-4-(5-ethoxypyrimidin-2-yl)-5-(hydroxy methyl)-1H-imidazol-1-yl}methyl]-biphenyl-2-yl]-1,2,4-oxadiazol-5(4H)-one,
methyl 2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid,
methyl 1-[{2′-(1H-tetrazol-5-yl)biphenyl-4-yl}methyl]-2-butyl-4-(5-ethoxypyrimidin-2-yl)-1H-imidazole-5-carboxylic acid,
2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
2-butyl-N-ethyl-4-(4-fluorophenyl)-1-[{2′-(5-oxo-4,5-dihydro-1,2,4-oxadiazol-3-yl)biphenyl-4-yl}methyl]-1H-imidazole-5-carboxamide, and
[1-[{2′-(1H-tetrazol-5-yl)biphenyl-4-yl}methyl]-2-butyl-4-(4-fluorophenyl)-1H-imidazol-5-yl](morpholino)methanone.

Although specific compounds of the 1-(biphenyl-4-yl-methyl)-1H-imidazole derivative represented by the formula (I) are given above, the compounds may be used either alone or in combination of each of them.

If the compound of the invention has geometrical isomers or optical isomers, the invention encompasses all of such isomers. Isolation of these isomers is carried out by an ordinary method.

Salts of the compound represented by the formula (I) are not particularly limited, if they are pharmaceutically acceptable salts. When the compound is processed as an acidic compound, a metal salt such as sodium salt, potassium salt, magnesium salt, and calcium salt; and a salt with an organic base such as trimethylamine, triethylamine, pyridine, picoline, N-methylpyrrolidine, N-methylpiperidine, and N-methylmorpholine can be mentioned. When the compound is processed as a basic compound, an acid addition salt and the like including a salt with a mineral acid, for example, hydrochloric acid salt, hydrobromic acid salt, hydroiodic acid salt, sulfuric acid salt, nitric acid salt, phosphoric acid salt, and the like; an organic acid addition salt, for example, benzoic acid salt, methanesulfonic acid salt, ethanesulfonic acid salt, benzene sulfonic acid salt, p-toluene sulfonic acid salt, maleic acid salt, fumaric acid salt, tartaric acid salt, citric acid salt, and acetic acid salt; or the like can be mentioned.

Examples of the solvate of the compound represented by the formula (I) or salt thereof include a hydrate and the like, but not limited thereto.

In addition, compounds which are metabolized in a living body and converted into the compounds represented by the aforementioned formula (I), so called prodrugs, all fall within the scope of the compounds of the invention. Examples of groups which form the prodrugs of the compounds of the invention include the groups described in “Progress in Medicine”, vol. 5, pp. 2157-2161, 1985, Life Science Medica, and the groups described in “Development of Drugs”, vol. 7, Molecular Designs, pp. 163 to 198, 1990, Hirokawa Shoten.

(Method for Producing Compounds Represented by the Formula (I), or Salts or a Solvates Thereof)

The compounds represented by the formula (I), or salts or solvates thereof can be produced according to various known methods, and the production method is not specifically limited. For example, the compounds can be produced according to the following reaction step. Further, when each reaction shown below is performed, functional groups other than the reaction sites may be protected beforehand as required, and deprotected in an appropriate stage. Furthermore, the reaction in each step may be performed by an ordinarily used method, and isolation and purification can be performed by a method suitably selected from conventional methods such as crystallization, recrystallization, chromatography, or the like, or a combination thereof.

Among the compounds represented by the formula (I) of the invention, the compound represented by the formula (Ia) can be produced according to the following method, but the production method is not limited thereto. Specifically, as illustrated in the Reaction Scheme 1 below, the compound (IV) is reacted with compound (V), the resulting compound represented by the formula (VI) is reacted with formalin, and subsequently the hydroxy group is oxidized by an appropriate oxidizing agent to give the compound (VIII). By reacting the compound (VIII) and the compound (IX) and reducing the formyl group with an appropriate reducing agent, the compound (XI) can be obtained. Further, the cyano group of the compound (XI) is reacted with hydroxylamine and condensed with an appropriate condensing agent to produce the compound represented by the formula (Ia) of the present invention.

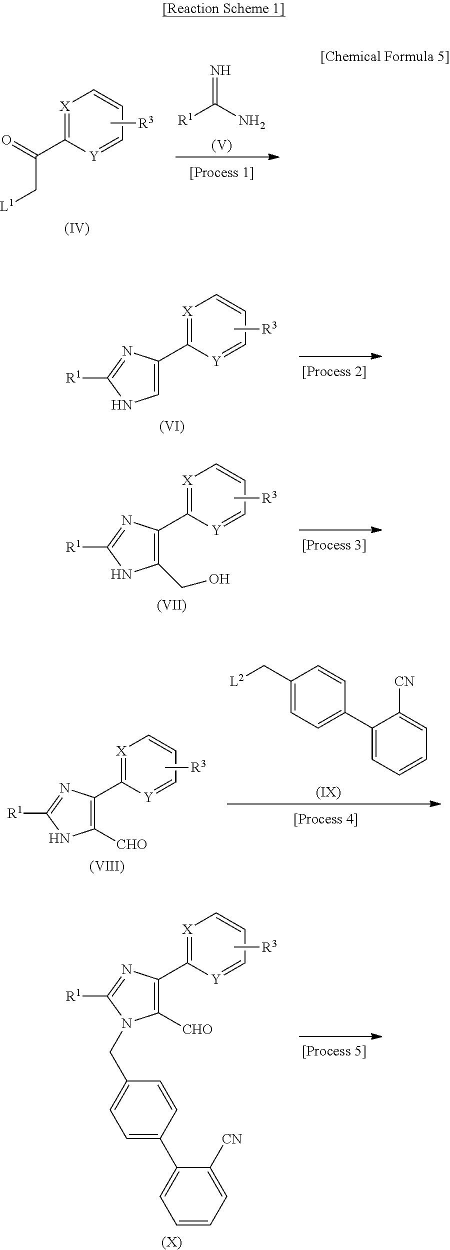

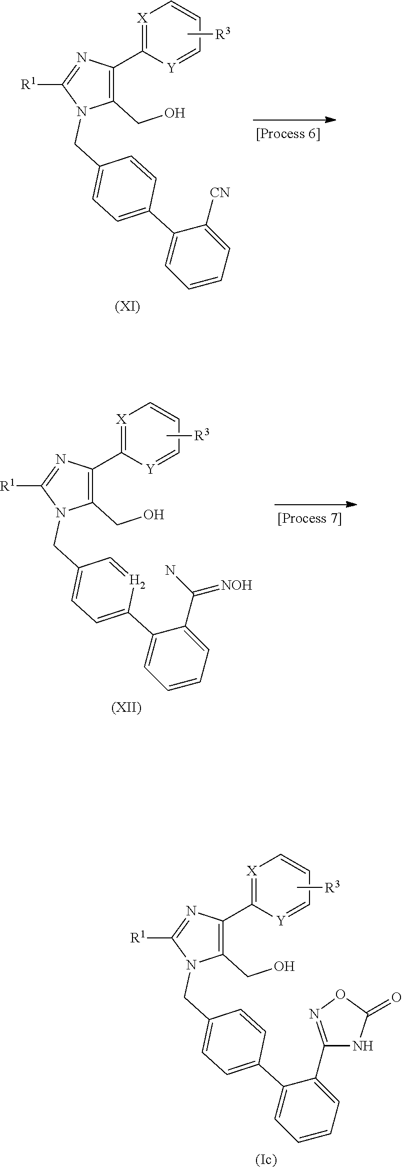

(in the formula, R¹, R³, X, and Y are as defined above, L¹ and L² represent a leaving group such as a halogen atom, a sulfonic ester substituted on a hydroxy group (methane sulfonic ester, trifluoromethanesulfonic ester, or the like), or the like.

### [Process 1]

The reaction between the ketone (IV) having a suitable leaving group L¹ at α position and the amidine (V) may be carried out in a solvent in the presence of a base. The solvent is not specifically limited, and ethyl acetate, isopropyl acetate, toluene, benzene, dioxane, tetrahydrofuran, acetonitrile, propionitrile, N,N-dimethylformamide, N-methylpyrrolidone, dimethylsulfoxide, or the like may be used either alone or in combination thereof. The base is not specifically limited, and the examples thereof include sodium hydride, potassium tert-butoxide, potassium carbonate, sodium carbonate, and the like. The amount of the base to be used is preferably about 1 to about 5 molar equivalent per the compound (IV). The reaction conditions may vary depending on the reaction materials used. However, the reaction is generally carried out at 0° C. to 150° C., and preferably 40° C. to 100° C. for 1 minute to 24 hours, and more preferably for 5 minutes to 18 hours to obtain the target compound (VI).

### [Process 2]

The imidazole (VI) can be hydroxymethylated according to a common method which uses formaldehyde with a base in a solvent, for example. The base is not specifically limited, and the examples thereof include a tertiary amine such as triethylamine, tributylamine, ethyldiisopropylamine, tetramethylenediamine, pyridine and the like; an alkali metal hydroxide such as lithium hydroxide, sodium hydroxide, potassium hydroxide, and the like; a metal carbonate salt such as potassium carbonate, sodium carbonate, cesium carbonate, and the like; and the like. The amount of the base to be used is preferably about 1 to about 5 molar equivalent per the compound (VI). Formaldehyde is not specifically limited, and a commercially available 35% aqueous solution can be used. The solvent is not specifically limited, and methanol, ethanol, 2-propanol, acetone, ethyl acetate, isopropyl acetate, toluene, benzene, dioxane, tetrahydrofuran, acetonitrile, propionitrile, N,N-dimethylformamide, N-methylpyrrolidone, dimethylsulfoxide, or the like may be used either alone or in combination thereof. The reaction conditions may vary depending on the reaction materials used. However, the reaction is generally carried out at 0° C. to 180° C., and preferably 80° C. to 150° C. for 1 to 48 hours, and more preferably for 8 to 24 hours to obtain the target compound (VII).

### [Process 3]

The alcohol (VII) can be oxidized in a solvent by using a suitable oxidizing agent. The oxidizing agent is not specifically limited, and a chromic acid-based oxidizing agent such as pyridinium chlorochromate and pyridinium dichromate; a ruthenium-based oxidizing agent such as tetrapropylammonium perruthenate; a hypervalent iodine compound such as 1,1,1-tri acetoxy-1,1-dihydro-1,2-benziodoxol-3(1H)-one; ammonium cerium (IV) nitrate; manganese dioxide; and the like. However, so called Swern oxidation condition using dimethylsulfoxide can be also used. The solvent is required to be inert to the oxidation condition, and dichloromethane, dichloroethane, chloroform, ethyl acetate, isopropyl acetate, acetic acid, toluene, benzene, dioxane, tetrahydrofuran, acetonitrile, propionitrile, or the like may be used either alone or in combination thereof. The reaction conditions may vary depending on the reaction materials or oxidizing agent used. However, the reaction is generally carried out at −20° C. to 100° C., and preferably 0° C. to 50° C. for 10 minutes to 24 hours, and more preferably for 20 minutes to 12 hours to obtain the target compound (VIII).

### [Process 4]

Alkylation of the compound (VIII) by using the compound (IX) is carried out in a solvent which has no influence on the reaction in the presence of a base. The base is not specifically limited, and examples thereof include an alkali metal salt such as potassium hydroxide, sodium hydroxide, sodium hydrogen carbonate, potassium carbonate and the like; an amine such as pyridine, triethylamine, N,N-dimethyl aniline, 1,8-diazabicyclo[5.4.0]undec-7-ene, and the like; a metal hydride such as potassium hydride, sodium hydride, and the like; an alkali metal alkoxide such as sodium methoxide, sodium ethoxide, potassium tert-butoxide, and the like; and the like. The amount of the base to be used is preferably about 1 to about 5 molar equivalent per the compound (VIII). The solvent is not specifically limited, and benzene, toluene, xylene, acetonitrile, propionitrile, tetrahydrofuran, 1,4-dioxane, diethyl ether, acetone, 2-butanone, chloroform, dichloromethane, N,N-dimethylformamide, dimethylsulfoxide, or the like may be used either alone or in combination thereof. The reaction conditions may vary depending on the reaction materials used. However, the reaction is generally carried out at 0° C. to 150° C., and preferably 20° C. to 120° C. for 5 minutes to 24 hours, and more preferably for 20 minutes to 12 hours to obtain the target compound (X).

### [Process 5]

The aldehyde (X) can be reduced in a solvent by using a suitable reducing agent. The reducing agent is not specifically limited, and the examples thereof include sodium borohydride, sodium cyanoborohydride, lithium triethylborohydride, lithium tri(sec-butyl)borohydride, potassium tri(sec-butyl)borohydride, lithium borohydride, zinc borohydride, sodium acetoxyborohydride, lithium aluminum hydride, and the like. The solvent is not specifically limited, and examples thereof include methanol, ethanol, propanol, diethyl ether, diisopropyl ether, tetrahydrofuran, acetonitrile, N,N-dimethylformamide, N,N-dimethylacetamide, dimethylsulfoxide, and the like, or a water-containing solvent thereof and the like. The reaction conditions may vary depending on the reaction materials used. However, the reaction is generally carried out at −70° C. to 80° C., and preferably −10° C. to 40° C. for 5 minutes to 24 hours, and more preferably for 10 minutes to 6 hours to obtain the target compound (XI).

### [Process 6]

The reaction between the cyano compound (XI) and hydroxylamine can be carried out in a solvent. The solvent is not specifically limited, and N,N-dimethylformamide, N,N-dimethylacetamide, N-methylpyrrolidone, dimethylsulfoxide, methanol, ethanol, isopropanol, 1,4-dioxane, tetrahydrofuran, or the like may be used either alone or in combination thereof. When an acid salt such as hydroxylamine hydrochloride, hydroxylamine sulfuric acid, hydroxylamine oxalic acid, and the like is used as hydroxylamine, a suitable base, for example, potassium carbonate, sodium hydrogen carbonate, sodium hydroxide, triethylamine, sodium methoxide, sodium hydride, and the like, is used in an equivalent amount or a slightly excess amount for the reaction. The reaction conditions may vary depending on the reaction materials used. However, the reaction is generally carried out at 0° C. to 180° C., and preferably 50° C. to 120° C. for 1 minute to 3 days, and more preferably for 1 hour to 24 hours to obtain the amide oxime (XII).

### [Process 7]

Conversion of the amide oxime (XII) to the compound (Ia) can be carried in a solvent in the presence of a base by using a carbonylation reagent. The solvent is not specifically limited, and 1,2-dichloroethane, chloroform, dichloromethane, ethyl acetate, isopropyl acetate, toluene, benzene, tetrahydrofuran, dioxane, acetonitrile, propionitrile, N,N-dimethylformamide, N,N-dimethylacetamide, N-methylpyrrolidone, diethyl ether, or the like may be used either alone or in combination thereof. The base is not specifically limited, and pyridine, DMAP, collidine, lutidine, DBU, DBN, DABCO, triethylamine, diisopropylethylamine, diisopropylpentylamine, trimethylamine, lithium carbonate, sodium carbonate, potassium carbonate, cesium carbonate, sodium hydrogen carbonate, potassium hydrogen carbonate, or the like may be used. The carbonylation reagent is not specifically limited, and N,N′-carbonyldiimidazole, triphosgene, methyl chloroformate, ethyl chloroformate, or the like can be used. The reaction conditions may vary depending on the reaction materials used. However, the reaction is generally carried out at 0° C. to 120° C., and preferably 10° C. to 80° C. for 5 min to 3 days, and more preferably for 1 hour to 12 hours to obtain the compound (Ia).

Further, among the compounds represented by the formula (I), the compound represented by the formula (Ib) can be produced according to the following method, but the production method is not limited thereto.

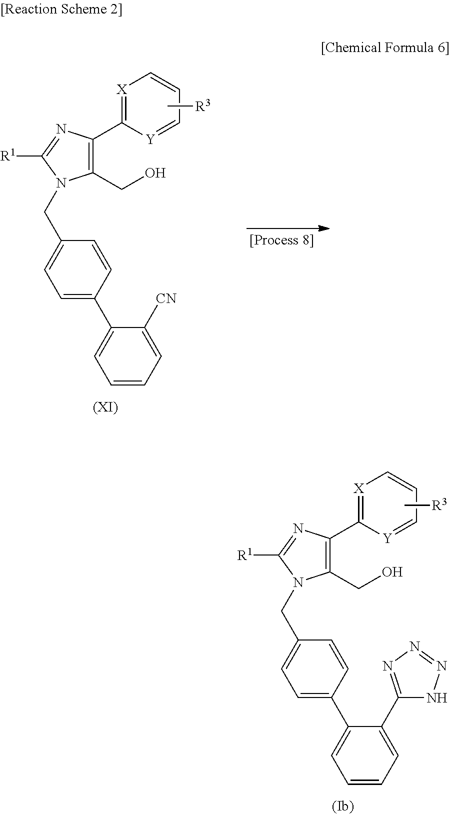

(in the formula, R¹, R³, X, and Y are as defined above).

### [Process 8]

The reaction between the cyano compound (XI) and an azide compound can be carried out in a solvent. Examples of the azide compound that can be used include trimethyltin azide, tributyltin azide, triphenyltin azide, sodium azide, hydrogen azide, and the like. Further, trimethylsilyl azide may be used in the presence of dibutyltin oxide. The solvent is not specifically limited, and methanol, ethanol, isopropanol, ethyl acetate, isopropyl acetate, toluene, benzene, dioxane, tetrahydrofuran, acetonitrile, propionitrile, N,N-dimethylformamide, N-methylpyrrolidone, dimethylsulfoxide, or the like may be used either alone or in combination thereof. The reaction conditions may vary depending on the reaction materials used. However, the reaction is generally carried out at 0° C. to 180° C., and preferably 50° C. to 120° C. for 1 minute to 2 weeks, and more preferably for 1 hour to 3 days to obtain the target compound.

Among the compounds that are represented by the formula (I) of the invention, the compound represented by the formula (Ic) or the formula (Id) can be produced according to the following method, but the production method is not limited thereto. Specifically, as illustrated in the Reaction Scheme 3 below, the carboxylic acid (XI) can be obtained by oxidizing the aldehyde (X). Further, by dehydration condensation of the carboxylic acid (XI) with alcohol or amine, the compound (XII) is obtained. Further, the cyano group of the compound (XII) is reacted with a hydroxylamine and condensed with an appropriate condensing agent to produce the compound represented by the formula (Ic) of the invention. Further, by hydrolyzing the compound (Ic), the compound represented by the formula (Id) of the invention can be produced.

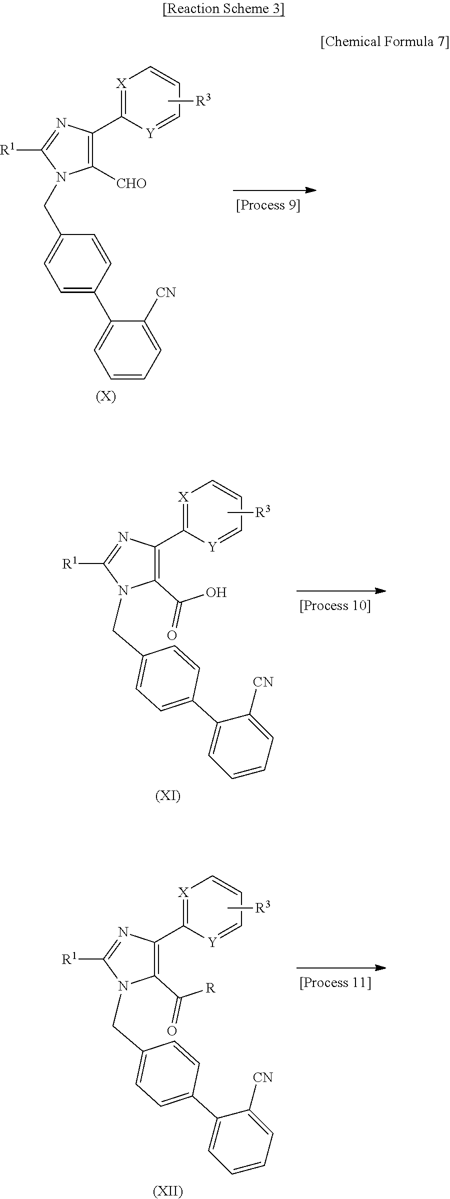

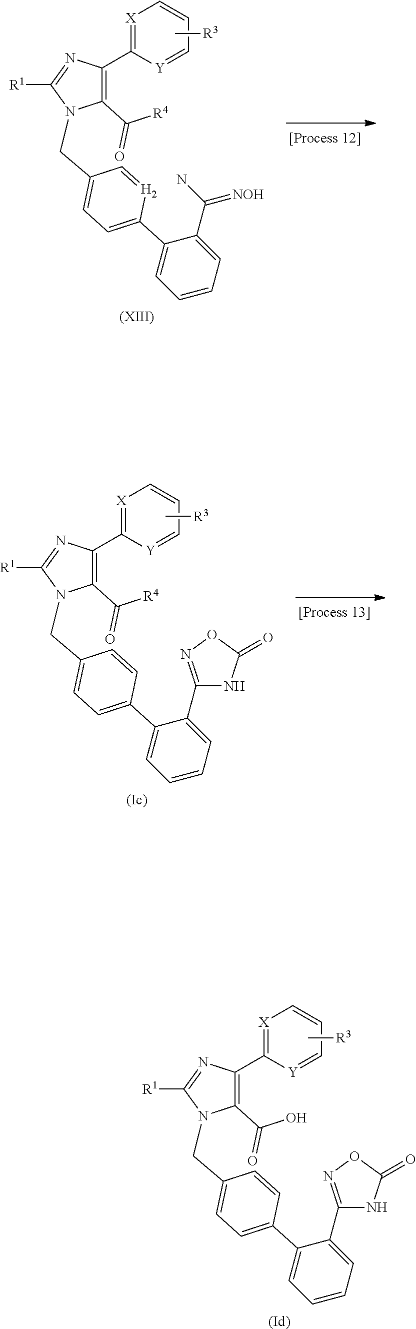

(in the formula, R¹, R³, R⁴, X, and Y are as defined above).

### [Process 9]

The aldehyde (X) can be oxidized in a solvent by using a suitable oxidizing agent. Examples of the oxidizing agent that can be used include potassium permanganate, manganese dioxide, silver oxide, hypochloric acid salt, and the like. Further, the oxidation can be carried out by using periodate salt having dichromate salt or anhydrous chromic acid as a catalyst. The solvent is not specifically limited, and acetone, methanol, ethanol, N,N-dimethylformamide, N-methylpyrrolidinone, 1,3-dimethyl-2-imidazolidinone, diethyl ether, tetrahydrofuran, 1,4-dioxane, dichloromethane, chloroform, carbon tetrachloride, pyridine, dimethylsulfoxide, acetonitrile, 2-propanol, tert-butanol, water, or the like may be used either alone or in combination thereof. When hypochloric acid salt is used as an oxidizing agent, in particular, a pH adjusting agent such as sodium dihydrogen phosphate or the like can be used. Further, by using an entrapping agent such as 2-methyl-2-butene or the like, by-products such as hypochlorous acid or the like generated in reaction system can be entrapped. The reaction conditions may vary depending on the reaction materials used. However, the reaction is generally carried out at 0° C. to 150° C., and preferably 10° C. to 100° C. for 1 minute to 1 week, and more preferably for 5 minutes to 1 day to obtain the target compound (XI).

### [Process 10]

The compound (XII) can be obtained by condensation of the carboxylic acid (XI) with alcohol or amine in a solvent. When the ester is produced according to the reaction with an alcohol, a suitable condensation reagent can be used in addition to the employment of an acid catalyst condition known as Fisher esterification reaction. The condensation reagent is not specifically limited, and an active esterification reagent such as 2,4,6-trichlorobenzoyl chloride and the like in addition to carbodiimide such as N,N′-dicyclohexylcarbodiimide, 1-ethyl-3-(3-dimethylaminopropyl)carbodiimide hydrochloric acid salt, and the like. Further, when methyl ester is synthesized, in particular, a methylation reagent such as diazo methane, trimethylsilyldiazomethane, and the like can be used. When an amide is synthesized by reaction with an amine, a suitable condensation reagent can be used in addition to the employment of so called Schotten-Baumann reaction condition in which the carboxylic acid (XI) is converted to acid chloride and reacted with an amine. The condensation reagent is not specifically limited, and carbodiimide such as N,N′-dicyclohexylcarbodiimide, 1-ethyl-3-(3-dimethylaminopropyl)carbodiimide hydrochloric acid salt, and the like. The solvent is not specifically limited as long as it is inert, and methylene chloride, chloroform, ether, tetrahydrofuran, dimethylformamide, dimethylacetamide, benzene, toluene, xylene, ethyl acetate, or the like may be used either alone or in combination thereof. The reaction conditions may vary depending on the reaction materials used. However, the reaction is generally carried out at 0° C. to 120° C., and preferably 0° C. to 80° C. for 1 minute to 1 week, and more preferably for 5 minutes to 3 days to obtain the target compound (XII).

### [Process 11 and Process 12]

The reaction of the compound (XII) to give the compound (Ic) can be carried out by applying the method described in Process 6 and Process 7.

### [Process 13]

The compound (Ic) can be converted into the compound (Id) by carrying out hydrolysis with a base particularly when R⁴ is an alkoxy group. The base is not specifically limited, and hydroxides such as sodium hydroxide, potassium hydroxide, lithium hydroxide, calcium hydroxide, cesium hydroxide, barium hydroxide, tetramethylammonium hydroxide, and the like; carbonates such as sodium carbonate, cesium carbonate, potassium carbonate, and the like. The solvent is not specifically limited, and methanol, ethanol, tetrahydrofuran, dioxane, chloroform, N,N-dimethylformamide, water, or the like may be used either alone or in combination thereof. The reaction conditions may vary depending on the reaction materials used. However, the reaction is generally carried out at 0° C. to 120° C., and preferably 0° C. to 80° C. for 1 minute to 1 week, and more preferably for 5 minutes to 3 days to obtain the target compound.

Further, among the compounds that are represented by the formula (I) of the invention, the compound represented by the formula (Ie) or the formula (If) can be produced by the following method, but the production method is not limited thereto. Specifically, as illustrated in the Reaction Scheme 4 below, the compound (Ie) can be obtained by reacting the cyano group of the compound (XII) with an azide compound. Further, by hydrolyzing the compound (Ie), the compound represented by the formula (If) of the invention can be produced. The Process 14 and Process 15 of the Reaction Scheme 4 can be carried out by applying the method described in Process 8 and Process 13 above.

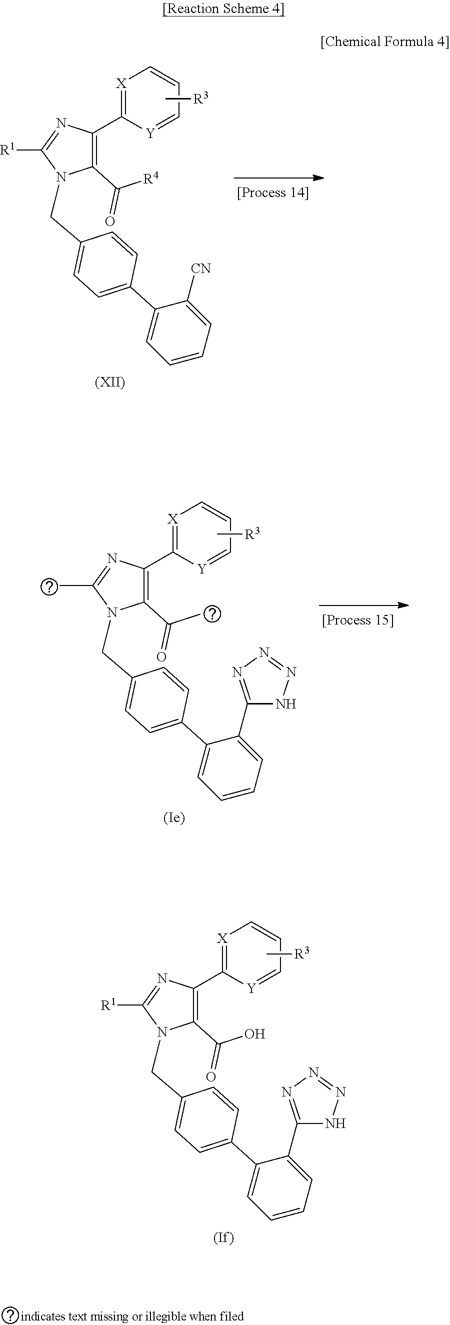

(in the formula, R¹, R³, R⁴, X, and Y are as defined above).

If necessary, the intermediates and target compounds that are obtained from each of the reaction above can be isolated and purified by a purification method that is generally used in a field of organic synthesis chemistry, e.g., filtration, extraction, washing, drying, concentration, recrystallization, various chromatographic methods, and the like. Furthermore, the intermediates may be used for the next reaction without any specific purification.

Various isomers may be isolated by applying a general method based on a difference in physicochemical properties among the isomers. For example, a racemic mixture may be resolved into an optically pure isomer by common racemic resolution such as optical resolution by which a diastereomer salt is formed with a common optically active acid such as tartaric acid or by a method of using optically active column chromatography. Further, a mixture of diastereomers can be resolved by fractional crystallization or various chromatographic methods, for example. Furthermore, an optically active compound can be also produced by using an appropriate starting compound that is optically active.

The compound (I) obtained may be converted into a salt according to a common method. Furthermore, it may be converted into a solvate with a solvent such as a solvent for reaction or a solvent for re-crystallization, or into a hydrate.

The compound (I) obtained may be converted into a salt according to a common method. Furthermore, it may be converted into a solvate with a solvent like a solvent for reaction or a solvent for recrystallization, or into a hydrate.

Examples of dosage form of the pharmaceutical composition containing the compounds of the invention, salts or a solvates thereof as an effective component include, for example, those for oral administration such as tablet, capsule, granule, powder, syrup, or the like and those for parenteral administration such as intravenous injection, intramuscular injection, suppository, inhalant, transdermal preparation, eye drop, nasal drop, or the like. In order to prepare a pharmaceutical preparation in the various dosage forms, the effective component may be used alone, or may be used in appropriate combination with other pharmaceutically acceptable carriers such as excipients, binders, extending agents, disintegrating agents, surfactants, lubricants, dispersing agents, buffering agents, preservatives, corrigents, perfumes, coating agents, diluents, and the like to give a pharmaceutical composition.

Although the administration amount of the pharmaceutical agent of the invention may vary depending on the weight, age, sex, symptoms, and the like of a patient, in terms of the compound represented by the general formula (I), generally 0.1 to 1000 mg, especially 1 to 300 mg, may be administered orally or parenterally at one time or several times as divided portions per day for an adult.

### EXAMPLES

Herein below, the invention will be explained in greater detail with reference to examples. However, the invention is not limited to these examples. The abbreviations used in the examples have the following definitions.
s: singlet
d: doublet
t: triplet
q: quartet
m: multiplet
br: broad
J: coupling constant
Hz: hertz
CDCl₃: deuterated chloroform
CD₃OD: deuterated methanol

¹H-NMR: proton nuclear magnetic resonance

### Example 1

### Production of 3-[4′-[{2-butyl-4-(4-fluorophenyl)-5-(hydroxymethyl)-1H-imidazol-1-yl}methyl]biphenyl-2-yl]-1,2,4-oxadiazol-5(4H)-one

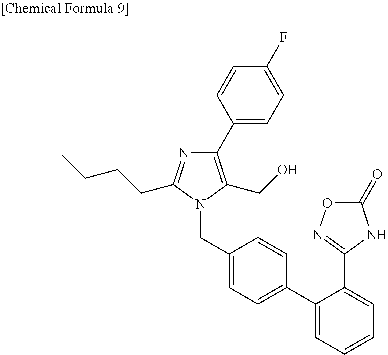

Process 1: A N,N-dimethylformamide (5.0 mL) solution of 2-bromo-4′-fluoro acetophenone (1.08 g, 5.0 mmol) was added dropwise to a N,N-dimethylformamide (30 mL) solution of pentaimidamide (1.36 g, 10.0 mmol) and potassium carbonate (2.08 g, 15.0 mmol) and stirred at 60° C. for 3 hours. The reaction mixture was added water and extracted with ethyl acetate. The organic layer was combined, washed with water and brine, dried over anhydrous sodium sulfate, and concentrated in vacuo. The residues obtained were subjected to silica gel column chromatography (chloroform/methanol=10:1), to obtain 2-butyl-4-(4-fluorophenyl)-1H-imidazole as a white solid (718.5 mg, 65.8).

¹H-NMR, (CDCl₃) δ: 0.90 (3H, t, J=7 Hz), 1.32-1.41 (2H, m), 1.65-1.74 (2H, m), 2.74 (2H, t, J=8 Hz), 7.01-7.06 (2H, m), 7.14 (1H, s), 7.64-7.67 (2H, m).

Process 2: 35% aqueous solution of formaldehyde (10 mL) was added dropwise to a 2-propanol (10 mL) and N,N-dimethylformamide (4.0 mL) solution of 2-butyl-4-(4-fluorophenyl)-1H-imidazole (412.8 mg, 1.89 mmol) and potassium carbonate (784.2 mg, 5.67 mmol) and stirred at 100° C. for 6 hours. After cooling, 35% aqueous solution of formaldehyde (2.0 mL) was added dropwise to the reaction mixture and stirred at 100° C. for 12 hours. The reaction mixture was added water and extracted with ethyl acetate. The organic layer was combined, washed with water and brine, dried over anhydrous sodium sulfate, and concentrated in vacuo. The residues obtained were subjected to silica gel column chromatography (chloroform/methanol=10:1) to obtain {2-butyl-4-(4-fluorophenyl)-1H-imidazol-5-yl}methanol as a white solid (246.2 mg, 52.5%).

¹H-NMR (CD₃OD) δ: 0.96 (3H, t, J=7 Hz), 1.36-1.43 (2H, m), 1.68-1.75 (2H, m), 2.71 (2H, t, J=8 Hz), 4.57 (2H, s), 7.11-7.16 (2H, m), 7.60-7.63 (2H, m).

Process 3: Manganese dioxide (2.19 mg, 25.2 mmol) was added to a 1,4-dioxane (7.0 ml) and dichloromethane (7.0 mL) solution of (2-butyl-4-(4-fluorophenyl)-1H-imidazol-5-yl)methanol (626.9 mg, 2.52 mmol) and stirred at 40° C. for 2 hours. After cooling, the mixture was filtered through a pad of celite followed by washing with chloroform and concentration in vacuo. The residues obtained were subjected to silica gel column chromatography (chloroform/methanol=10:1) to obtain 2-butyl-4-(4-fluorophenyl)-1H-imidazole-5-carbaldehyde as a white solid (518.6 mg, 83.6%).

¹H-NMR (CDCl₃) δ: 0.94 (3H, t, J=7 Hz), 1.36-1.46 (2H, m), 1.75-1.83 (2H, m), 2.85 (2H, t, J=8 Hz), 7.15-7.19 (2H, m), 7.70-7.75 (2H, m), 9.72 (1H, s), 11.36 (1H, br).

Process 4: A N,N-dimethylformamide (3.0 mL) solution of 4′-bromomethyl-2-cyanobiphenyl (152.8 g, 0.843 mmol) was added dropwise to a N,N-dimethylformamide (3.0 mL) suspension of 2-butyl-4-(4-fluorophenyl)-1H-imidazole-5-carbaldehyde (138.3 mg, 0.562 mmol) and potassium carbonate (116.5 mg, 0.843 mmol) and stirred at 60° C. for 4 hours. The reaction mixture was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=1:1) to obtain 4′-[{2-butyl-4-(4-fluorophenyl)-5-formyl-1H-imidazol-1-yl}methyl]biphenyl-2-carbonitrile as a pale white amorphous (229.1 mg, 93.2%).

¹H-NMR (CDCl₃) δ: 0.92 (3H, t, J=7 Hz), 1.37-1.46 (2H, m), 1.72-1.79 (2H, m), 2.77 (2H, t, J=8 Hz), 5.72 (2H, s), 7.14-7.19 (2H, m), 7.21-7.23 (2H, m), 7.42-7.49 (2H, m), 7.53-7.55 (2H, m), 7.61-7.76 (4H, m), 9.78 (1H, s).

Process 5: Sodium borohydride (26.2 mg, 0.694 mmol) was added to a methanol (3.0 mL) solution of 4′-[{2-butyl-4-(4-fluorophenyl)-5-formyl-1H-imidazol-1-yl}methyl]biphenyl-2-carbonitrile (60.7 mg, 0.139 mmol) under ice cooling and stirred at room temperature for 3 hours. After concentration in vacuo, the ethyl acetate was added, washed with an aqueous solution of ammonium chloride, water, and brine, and then dried over anhydrous sodium sulfate followed by concentration in vacuo. The residues obtained were subjected to silica gel column chromatography (chloroform/methanol=10:1) to obtain 4′-[{2-butyl-4-(4-fluorophenyl)-5-(hydroxymethyl)-1H-imidazol-1-yl}methyl]biphenyl-2-carbonitrile as a white amorphous (48.8 mg, 79.9%).

¹H-NMR (CDCl₃) δ: 0.86 (3H, t, J=7 Hz), 1.30-1.39 (2H, m), 1.60-1.68 (2H, m), 2.62 (2H, t, J=8 Hz), 4.55 (2H, s), 5.25 (2H, s), 7.02-7.10 (4H, m), 7.43-7.52 (4H, m), 7.61-7.66 (3H, m), 7.78 (2H, d, J=8 Hz).

Process 6: Dimethylsulfoxide (1 mL) and sodium hydrogen carbonate (173.2 mg, 2.06 mmol) were added to hydroxylamine hydrochloride (121.6 mg, 1.75 mmol) and stirred at 40° C. for 1 hour. Dimethylsulfoxide (0.5 mL) solution of 4′-[{2-butyl-4-(4-fluorophenyl)-5-(hydroxymethyl)-1H-imidazol-1-yl}methyl]biphenyl-2-carbonitrile (45.3 mg, 0.103 mmol) was added to the reaction mixture and stirred at 90° C. for 24 hours. After completion of the reaction, the reaction mixture was added water and extracted with ethyl acetate. The organic layer was combined, washed with water and brine, dried over anhydrous sodium sulfate, and concentrated in vacuo. The residues obtained were subjected to silica gel column chromatography (chloroform/methanol=10:1) to obtain 4′-[{2-butyl-4-(4-fluorophenyl)-5-(hydroxymethyl)-1H-imidazol-1-yl}methyl]-N′-hydroxybiphenyl-2-carboimidamide as a pale yellow solid (35.1 mg, 72.1%).

¹H-NMR (CDCl₃) δ: 0.88 (3H, t, J=7 Hz), 1.33-1.42 (2H, m), 1.64-1.72 (2H, m), 2.69 (2H, t, J=8 Hz), 4.48 (2H, s), 4.50 (2H, s), 5.22 (2H, s), 7.03-7.07 (4H, m), 7.33-7.48 (5H, m), 7.52-7.54 (1H, m), 7.62-7.66 (2H, m).

Process 7: N,N′-carbonyldiimidazole (24.2 mg, 0.149 mmol) and 1,8-diazabicyclo[5.4.0]undec-7-ene (22.7 mg, 0.149 mmol) were added to a N,N-dimethylformamide (2.0 mL) solution of 4′-[{2-butyl-4-(4-fluorophenyl)-5-(hydroxymethyl)-1H-imidazol-1-yl}methyl]-N′-hydroxybiphenyl-2-carboimidamide (35.1 mg, 0.074 mmol) and stirred at room temperature for 3 hours. After completion of the reaction, the reaction mixture was added water and extracted with ethyl acetate. The organic layer was combined, washed with water and brine, dried over anhydrous sodium sulfate, and concentrated in vacuo. The residues obtained were purified and isolated by silica gel column chromatography (chloroform/methanol=10:1) to obtain the target compound as a pale yellow amorphous (12.6 mg, 34.1%).

¹H-NMR (CDCl₃) δ: 0.86 (3H, t, J=7 Hz), 1.23-1.34 (2H, m), 1.54-1.61 (2H, m), 2.37 (2H, t, J=8 Hz), 4.37 (2H, s), 5.17 (2H, s), 6.87-6.96 (4H, m), 7.20-7.22 (2H, m), 7.25-7.32 (3H, m), 7.41-7.44 (1H, m), 7.52-7.61 (2H, m).

### Example 2

### Production of methyl 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid

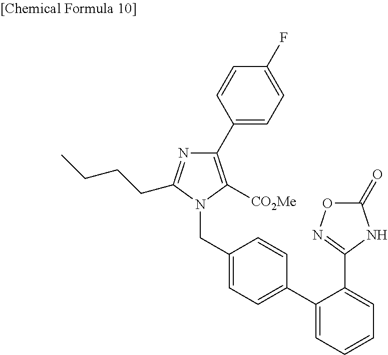

Process 1: 2-methyl-2-butene (270.0 mg, 3.85 mmol) and sodium dihydrogen phosphate (230.9 mg, 1.93 mmol) were added to a mixture solution of water (1.0 mL) and tert-butanol (4.0 mL) of the 4′-[{2-butyl-4-(4-fluorophenyl)-5-formyl-1H-imidazol-1-yl}methyl]biphenyl-2-carbonitrile (168.4 mg, 0.385 mmol) obtained in the Process 4 of the Example 1 and stirred at room temperature for 10 min. Sodium chlorite (104.5 mg, 1.16 mmol) was added to the reaction mixture and stirred at room temperature for 1 hour. The reaction mixture was added hydrochloric acid and extracted with ethyl acetate. The organic layer was combined, washed with brine, dried over anhydrous sodium sulfate, and concentrated in vacuo to obtain 2-butyl-1-{(2′-cyanobiphenyl-4-yl)methyl}-4-(4-fluorophenyl)-1H-imidazole-5-carboxylic acid as a crude product. The crude product obtained was used for the next process without any purification.

Process 2: The crude product of 2-butyl-1-{(2′-cyanobiphenyl-4-yl)methyl}-4-(4-fluorophenyl)-1H-imidazole-5-carboxylic acid obtained from the above was dissolved in methanol (3.0 mL), added a solution of trimethylsilyldiazomethane (2.0 mol/L in Et₂O, 288.7 μL, 0.578 mmol) and stirred at room temperature for 12 hours. The reaction mixture was added acetic acid to quench trimethylsilyl diazomethane, diluted with water and extracted with ethyl acetate. The organic layer was combined, washed with brine, dried over anhydrous sodium sulfate, and concentrated in vacuo. The residues obtained were subjected to silica gel column chromatography (chloroform/methanol=20:1) to obtain methyl 2-butyl-1-{(2′-cyanobiphenyl-4-yl)methyl}-4-(4-fluorophenyl)-1H-imidazole-5-carboxylic acid as a white amorphous (104.2 mg, 59.7%).

¹H-NMR (CDCl₃) δ: 0.91 (3H, t, J=7 Hz), 1.36-1.45 (2H, m), 1.66-1.78 (2H, m), 2.74 (2H, t, J=8 Hz), 3.66 (3H, s), 5.64 (2H, s), 7.06-7.12 (2H, m), 7.15-7.20 (2H, m), 7.42-7.55 (4H, m), 7.62-7.68 (3H, m), 7.75-7.77 (1H, m).

Process 3: Methyl 2-butyl-4-(4-fluorophenyl)-1-[{2′-(N′-(hydroxycarbamimidoyl) biphenyl-4-yl}methyl]-1H-imidazole-5-carboxylic acid was obtained as a pale yellow amorphous (63.4%) according to the same reaction and treatment as the Process 6 of the Example 1 by using methyl 2-butyl-1-{(2′-cyanobiphenyl-4-yl)methyl}-4-(4-fluorophenyl)-1H-imidazole-5-carboxylic acid instead of the 4′-[{2-butyl-4-(4-fluorophenyl)-5-(hydroxymethyl)-1H-imidazol-1-yl}ethyl]biphenyl-2-carbonitrile.

¹H-NMR (CDCl₃) δ: 0.89 (3H, t, J=7 Hz), 1.33-1.43 (2H, m), 1.63-1.74 (2H, m), 2.72 (2H, t, J=8 Hz), 3.65 (3H, s), 4.42 (2H, s), 5.60 (2H, s), 7.05-7.12 (4H, m), 7.34-7.41 (2H, m), 7.44-7.48 (3H, m), 7.55-7.57 (1H, m), 7.63-7.67 (2H, m).

Process 4: The target compound was obtained as a pale yellow amorphous (57.8%) according to the same reaction and treatment as the Process 7 of the Example 1 by using methyl 2-butyl-4-(4-fluorophenyl)-1-[{2′(N′(hydroxycarbamimidoyl)biphenyl-4-yl}methyl]-1H-imidazole-5-carboxylic acid instead of the 4′-[{2-butyl-4-(4-fluorophenyl)-5-(hydroxymethyl)-1H-imidazol-1-yl}methyl]-N′-hydroxybiphenyl-2-carboimidamide.

¹H-NMR (CDCl₃) δ: 0:92 (3H, t, J=7 Hz), 1.32-1.41 (2H, m), 1.64-1.71 (2H, m), 2.44 (2H, t, J=8 Hz), 3.55 (3H, s), 5.54 (2H, s), 6.91 (2H, t, J=9 Hz), 7.01 (2H, d, J=8 Hz), 7.27-7.37 (5H, m), 7.48 (1H, t, J=8 Hz), 7.56-7.64 (2H, m).

### Example 3

### Production of 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid

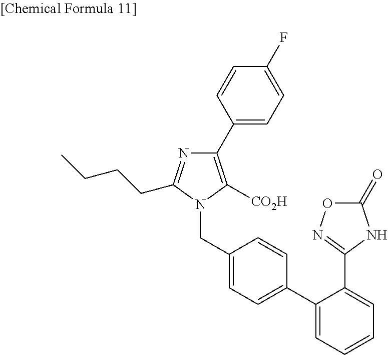

A water (0.5 mL) solution of lithium hydroxide-hydrate (12.0 mg, 0.287 mmol) was added dropwise to the methanol (2.0 mL) and water (0.5 mL) solution of methyl 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid (30.2 mg, 0.057 mmol) obtained in the Example 2, and stirred at room temperature for 3 hours and at 40° C. for 24 hours. The reaction mixture was added hydrochloric acid and extracted with ethyl acetate. The organic layer was combined, washed with water and brine, dried over anhydrous sodium sulfate, and concentrated in vacuo. The residues obtained were purified and isolated by silica gel column chromatography (chloroform/methanol=20:1) to obtain the target compound as a pale yellow amorphous (19.0 mg, 65.0%).

¹H-NMR (CD₃OD) δ: 0.89 (3H, t, J=8 Hz), 1.32-1.41 (2H, m), 1.55-1.63 (2H, m), 2.79 (2H, t, J=8 Hz), 5.78 (2H, s), 7.14 (2H, t, J=9 Hz), 7.21 (2H, d, J=8 Hz), 7.34 (2H, d, J=8 Hz), 7.50-7.55 (2H, m), 7.63-7.68 (2H, m).

### Example 4

### Production of 3-[4′-[{2-butyl-4-(5-ethoxypyrimidin-2-yl)-5-(hydroxymethyl)-1H-imidazol-1-yl}methyl]-biphenyl-2-yl]-1,2,4-oxadiazol-5(4H)-one

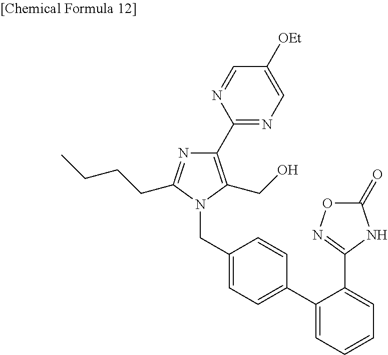

Process 1: 1,10-phenanthroline (180.2 mg, 1.0 mmol), copper (I) iodide (85.2 mg, 0.5 mmol), and cesium carbonate (2.44 g, 7.5 mmol) were added to an ethanol (10 mL) and toluene (10 mL) solution of 4-bromo-2-chloropyrimidine (967.1 mg, 5.0 mmol), and stirred at 100° C. for 3 hours. The reaction mixture was filtered and concentrated in vacuo. The residues obtained were subjected to silica gel column chromatography (hexane/ethyl acetate=1:1) to obtain 2-chloro-5-ethoxypyrimidine as a pale yellow solid (781.5 mg, 98.6%).

¹H-NMR (CDCl₃) δ: 1.43 (3H, t, J=7 Hz), 4.40 (2H, q, J=7 Hz), 8.52 (2H, s).

Process 2: Under argon atmosphere 1,4-dioxane (25 mL) and tributyl(1-ethoxy vinyl)tin (2.71 g, 7.5 mmol) were added to 2-chloro-5-ethoxypyrimidine (793.0 mg, 5.0 mmol), bis(tri-tert-butyl phosphine)palladium(0) (76.5 mg, 0.15 mmol), and cesium fluoride (1.67 g, 11 mmol) and stirred at 100° C. for 20 hours. The reaction mixture was filtered through a pad of celite and concentrated in vacuo. The residues obtained were subjected to silica gel column chromatography (hexane/ethyl acetate=1:1) to obtain 5-ethoxy-2-(1-ethoxy vinyl)pyrimidine as a pale Yellow solid (617.6 mg, 63.6%).

¹H-NMR (CDCl₃) δ: 1.40-1.46 (6H, m), 3.92 (2H, q, J=7 Hz), 4.23 (1H, d, J=3 Hz), 4.44 (28, q, J-=7 Hz), 4.57 (1H, d, J=3 Hz), 8.70 (2H, s).

Process 3: N-bromosuccinimide (565.9 mg, 3.18 mmol) was added to a tetrahydrofuran (9.0 mL) and water (3.0 mL) solution of 5-ethoxy-2-(1-ethoxyvinyl)pyrimidine (617.6 mg, 3.18 mmol) under ice cooling and stirred at room temperature for 10 min. The reaction mixture was added water and extracted with ethyl acetate. The organic layer was combined, washed with brine, dried over anhydrous sodium sulfate, and concentrated in vacuo. The residues obtained were subjected to silica gel column chromatography (hexane/ethyl acetate=1:1) to obtain 2-bromo-1-(5-ethoxypyrimidin-2-yl)ethanone as a white solid (708.6 mg, 90.9%).

¹H-NMR (CDCl₃) δ: 1.48 (3H, t, J=7 Hz), 4.34 (2H, s), 4.55 (2H, q, J=7 Hz), 9.10 (2H, s).

Process 4: 2-(2-butyl-1H-imidazol-4-yl)-5-ethoxypyrimidine was obtained as a white solid (28.8%) according to the same reaction and treatment as the Process 1 of the Example 1 by using 2-bromo-1-(5-ethoxypyrimidin-2-yl)ethanone instead of the 2-bromo-4′-fluoroacetophenone.

¹H-NMR (CDCl₃) δ: 0.92 (3H, t, J=7 Hz), 1.34-1.45 (5H, m), 1.69-1.76 (2H, m), 2.77 (2H, t, J=8 Hz), 4.44 (2H, q, J=7 Hz), 7.20 (1H, s), 8.83 (2H, s).

Process 5: {2-butyl-4-(5-ethoxypyrimidin-2-yl)-1H-imidazol-5-yl}methan of was obtained as a white solid (91.3%) according to the same reaction and treatment as the Process 2 of the Example 1 by using 2-(2-butyl-1H-imidazol-4-yl)-5-ethoxypyrimidine instead of the 2-butyl-4-(4-fluorophenyl)-1H-imidazole.

¹H-NMR (CD₃OD) δ: 0.96 (3H, t, J=7 Hz), 1.35-1.44 (5H, m), 1.68-1.76 (2H, m), 2.73 (2H, t, J=8 Hz), 4.47 (2H, q, J=7 Hz), 8.79 (2H, s).

Process 6: 2-butyl-4-(5-ethoxypyrimidin-2-yl)-1H-imidazole-5-carbaldehyde was obtained as a white solid (86.6%) according to the same reaction and treatment as the Process 3 of the Example 1 by using (2-butyl-4-(5-ethoxypyrimidin-2-yl)-1H-imidazol-5-yl)methanol instead of the {2-butyl-4-(4-fluorophenyl)-1H-imidazol-5-yl}methanol.

¹H-NMR (CDCl₃) δ: 0.97 (3H, t, J=7 Hz), 1.39-1.49 (5H, m), 1.76-1.84 (2H, m), 2.85 (2H, t, J=8 Hz), 4.50 (2H, q, J=7 Hz), 8.88 (2H, s), 9.75 (1H, s), 10.08 (1H, br).

Process 7: 4′-[{2-butyl-4-(5-ethoxypyrimidin-2-yl)-5-formyl-1H-imidazol-1-yl}methyl]biphenyl-2-carbonitrile was obtained as a pale yellow amorphous (95.2%) according to the same reaction and treatment as the Process 4 of the Example 1 by using {2-butyl-4-(5-ethoxypyrimidin-2-yl)-1H-imidazole-5-carbaldehyde instead of the 2-butyl-4-(4-fluorophenyl)-1H-imidazole-5-carbaldehyde.

¹H-NMR (CDCl₃) δ: 0.93 (38, t, J=7 Hz), 1.38-1.49 (5H, m), 1.72-1.80 (2H, m), 2.78 (2H, t, J=8 Hz), 4.50 (2H, q, J=7 Hz), 5.71 (2H, s), 7.22 (2H, d, J=8 Hz), 7.44-7.50 (2H, m), 7.55 (2H, d, J=8 Hz), 7.65 (1H, t, J=8 Hz), 7.77 (1H, d, J=8 Hz), 8.85 (2H, s), 9.77 (1H, s).

Process 8: 4′-[{2-butyl-4-(5-ethoxypyrimidin-2-yl)-5-(hydroxymethyl)-1H-imidazol-1-yl}methyl]biphenyl-2-carbonitrile was obtained as a white amorphous (82.0%) according to the same reaction and treatment as the Process 5 of the Example 1 by using 4′-[{2-butyl-4-(5-ethoxypyrimidin-2-yl)-5-formyl-1H-imidazol-1-yl}methyl]biphenyl-2-carbonitrile instead of the 4′-[{2-butyl-4-(4-fluorophenyl)-5-formyl-1H-imidazol-1-yl}methyl]biphenyl-2-carbonitrile.

¹H-NMR (CDCl₃) δ:

0.86 (3H, t, J=7 Hz), 1.30-1.43 (5H, m), 1.58-1.66 (2H, m), 2.62 (2H, t, J=8 Hz), 4.43 (2H, q, J=7 Hz), 4.53 (2H, s), 5.35 (2H, s), 7.14 (2H, d, J=8 Hz), 7.44-7.50 (2H, m), 7.53 (2H, d, J=8 Hz), 7.65 (1H, t, J=8 Hz), 7.76 (1H, d, J=8 Hz), 8.69 (2H, s).

Process 9: 4′-[{2-butyl-4-(5-ethoxypyrimidin-2-yl)-5-(hydroxymethyl)-1H-imidazol-1-yl}methyl]-N′-hydroxybiphenyl-2-carboimidamide was obtained as a white amorphous (85.8%) according to the same reaction and treatment as the Process 6 of the Example 1 by using 4′-[{2-butyl-4-(5-ethoxypyrimidin-2-yl)-5-(hydroxymethyl)-1 H-imidazol-1-yl}methyl]biphenyl-2-carbonitrile instead of the 4′-[{2-butyl-4-(4-fluorophenyl)-5-(hydroxymethyl)-1H-imidazol-1-yl}methyl]biphenyl-2-carbonitrile.

¹H-NMR (CDCl₃) δ: 0.86 (3H, t, J=7 Hz), 1.27-1.39 (2H, m), 1.41 (3H, t, J=7 Hz), 1.59-1.66 (2H, m), 2.63 (2H, t, J=8 Hz), 4.38-4.36 (4H, m), 4.54 (2H, s), 5.21 (2H, s), 7.02 (2H, d, J=8 Hz), 7.31-7.45 (5H, m), 7.50-7.51 (1H, m), 8.69 (2H, s).

Process 10: The target compound was obtained as a pale yellow amorphous (38.9%) according to the same reaction and treatment as the Process 7 of the Example 1 by using 4′-[{2-butyl-4-(5-ethoxypyrimidin-2-yl)-5-(hydroxymethyl)-1H-imidazol-1-yl}methyl]-N′-hydroxybiphenyl-2-carboimidamide instead of the 4′-[{2-butyl-4-(4-fluorophenyl)-5-(hydroxymethyl)-1H-imidazol-1-yl}methyl]-N′-hydroxybiphenyl-2-carboimidamide.

¹H-NMR (CDCl₃) δ: 0.82 (3H, t, J=7 Hz), 1.18-1.31 (5H, m), 1.50-1.56 (2H, m), 2.51 (2H, t, J=8 Hz), 4.28 (2H, q, J=7 Hz), 4.39 (2H, s), 5.18 (2H, s), 6.98 (2H, d, J=8 Hz), 7.22 (2H, d, J=8 Hz), 7.37 (1H, d, J=8 Hz), 7.45 (1H, t, J=8 Hz), 7.58 (1H, t, J=8 Hz), 7.68 (1H, d, J=8 Hz), 8.56 (2H, s).

### Example 5

### Production of [1-[{2′-(1H-tetrazol-5-yl)biphenyl-4-yl}methyl]-2-butyl-4-(5-ethoxypyrimidin-2-yl)-1H-imidazol-5-yl]methanol

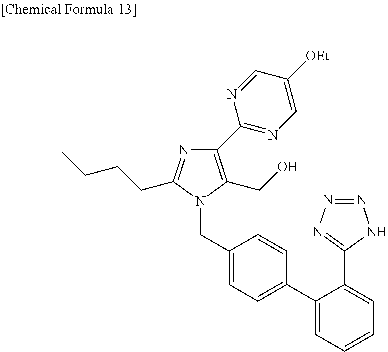

Under argon atmosphere, trimethylsilyl azide (1.78 mL, 13.6 mmol) and dibutyltin oxide (22.6 mg, 0.091 mmol) were added to the toluene (4.0 mL) solution of the 4′-[{2-butyl-4-(5-ethoxypyrimidin-2-yl)-5-(hydroxymethyl)-1H-imidazol-1-yl}methyl]biphenyl-2-carbonitrile (89.9 mg, 0.183 mmol) obtained in the Process 8 of the Example 4 and stirred at 95° C. for 18 hours. The reaction mixture was distilled off and the residues obtained were purified and isolated by silica gel column chromatography (chloroform/methanol=20:1) to obtain the target compound as a pale yellow amorphous (17.3 mg, 18.6%).

¹H-NMR (CDCl₃) δ: 0.77 (3H, t, J=7 Hz), 1.20-1.31 (5H, m), 1.44-1.51 (2H, m), 2.49 (2H, t, J=8 Hz), 4.32 (2H, q, J=7 Hz), 4.45 (2H, s), 5.15 (2H, s), 6.82-6.85 (4H, m), 7.27-7.30 (2H, m), 7.37-7.49 (2H, m), 7.69-7.71 (2H, m), 8.56 (2H, s).

### Example 6

### Production of methyl 2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid

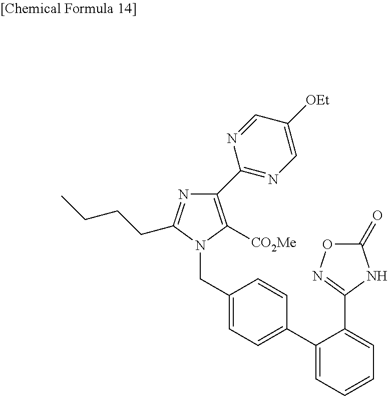

Process 1: Methyl 2-butyl-1-{(2′-cyanobiphenyl-4-yl)methyl}-4-(5-ethoxypyrimidin-2-yl)-1H-imidazole-5-carboxylic acid was obtained as a white amorphous (57.6) according to the same reaction and treatment as the Process 1 and 2 of the Example 2 by using the 4′-[{2-butyl-4-(5-ethoxypyrimidin-2-yl)-5-formyl-1H-imidazol-1-yl}methyl]biphenyl-2-carbonitrile obtained in the Process 7 of the Example 4 instead of the 4′-[{2-butyl-4-(4-fluorophenyl)-5-formyl-1H-imidazol-1-yl}methyl]biphenyl-2-carbonitrile.

¹H-NMR (CDCl₃) δ: 0.92 (3H, t, J=7 Hz), 1.39-1.48 (5H, m), 1.69-1.79 (2H, m), 2.75 (2H, t, J=8 Hz), 3.72 (3H, s), 4.49 (2H, q, J=7 Hz), 5.68 (2H, s), 7.17 (2H, d, J=8 Hz), 7.43-7.59 (5H, m), 7.62-7.67 (1H, m), 7.76-7.78 (1H, m), 8.82 (2H, s).

Process 2: Methyl 2-butyl-4-(5-ethoxypyrimidin-2-yl)-1-[{2′-(N′-hydroxycarbamimidoyl)biphenyl-4-yl}methyl]-1H-imidazole-5-carboxylic acid was obtained as a pale yellow amorphous (34.8%) according to the same reaction and treatment as the Process 6 of the Example 1 by using methyl 2-butyl-1-[{2′-cyanobiphenyl-4-yl}methyl]-4-(5-ethoxypyrimidin-2-yl)-1H-imidazole-5-carboxylic acid instead of the 4′-[{2-butyl-4-(4-fluorophenyl)-5-(hydroxymethyl)-1H-imidazol-1-yl}methyl]biphenyl-2-carbonitrile.

¹H-NMR (CDCl₃) δ: 0.90 (3H, t, J=7 Hz), 1.35-1.48 (5H, m), 1.64-1.76 (2H, m), 2.73 (2H, t, J=8 Hz), 3.71 (3H, s), 4.44 (2H, s), 4.48 (2H, q, J=7 Hz), 5.64 (2H, s), 7.09 (2H, d, J=8 Hz), 7.34-7.39 (2H, m), 7.45-7.49 (3H, m), 7.55-7.58 (1H, m), 8.80 (2H, s).

Process 3: The target compound was obtained as a pale yellow amorphous (90.8%) according to the same reaction and treatment as the Process 7 of the Example 1 by using methyl 2-butyl-4-(5-ethoxypyrimidin-2-yl)-1-[{2′-(N′-hydroxycarbamimidoyl)biphenyl-4-yl}methyl]-1H-imidazole-5-carboxylic acid instead of the 4′-[{2-butyl-4-(4-fluorophenyl)-5-(hydroxymethyl)-1H-imidazol-1-yl}methyl]-N′-hydroxybiphenyl-2-carboimidamide.

¹H-NMR (CDCl₃) δ: 0.91 (3H, t, J=7 Hz), 1.34-1.45 (5H, m), 1.67-1.74 (2H, m), 2.64 (2H, t, J=8 Hz), 3.65 (3H, s), 4.45 (2H, q, J=7 Hz), 5.59 (2H, s), 7.05 (2H, d, J=8 Hz), 7.27-7.29 (2H, m), 7.39 (1H, d, J=7 Hz), 7.45-7.49 (1H, m), 7.57-7.61 (1H, m), 7.69-7.71 (1H, m), 8.66 (2H, s).

### Example 7

### Production of 2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid

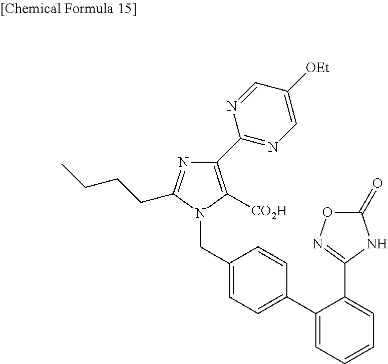

The target compound was obtained as a pale yellow amorphous (39.1%) according to the same reaction and treatment as the Process 1 of the Example 3 by using the methyl 2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid obtained in the Example 6 instead of the methyl 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid.

¹H-NMR (CD₃OD) δ: 0.89 (3H, t, J=7 Hz), 1.32-1.43 (5H, m), 1.57-1.64 (2H, m), 2.74 (2H, t, J=8 Hz), 4.48 (2H, q, J=7 Hz), 5.79 (2H, s), 7.17 (2H, d, J=7 Hz), 7.32 (2H, d, J=8 Hz), 7.49-7.54 (2H, m), 7.62-7.67 (2H, m), 8.83 (2H, s).

### Example 8

### Production of methyl 1-[{2′-(1H-tetrazol-5-yl)biphenyl-4-yl}methyl]-2-butyl-4-(5-ethoxypyrimidin-2-yl)-1H-imidazole-5-carboxylic acid

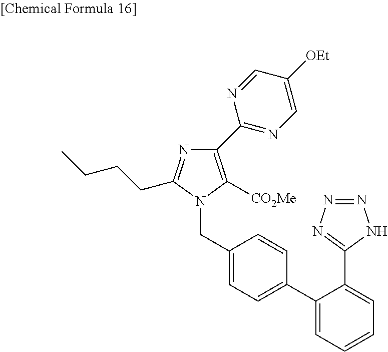

The target compound was obtained as a pale yellow amorphous (79.0%) according to the same reaction and treatment as the Process 1 of the Example 5 by using the methyl 2-butyl-1-{(2′-cyanobiphenyl-4-yl)methyl}-4-(5-ethoxypyrimidin-2-yl)-1H-imidazole-5-carboxylic acid obtained in the Process 1 of the Example 6 instead of the 4′-[{2-butyl-4-(5-ethoxypyrimidin-2-yl)-5-(hydroxymethyl)-1H-imidazol-1-yl}methyl]biphenyl-2-carbonitrile.

¹H-NMR (CDCl₃) δ: 0.90 (3H, t, J=7 Hz), 1.30-1.41 (5H, m), 1.61-1.69 (2H, m), 2.50 (2H, t, J=8 Hz), 3.65 (3H, s), 4.41 (2H, q, J=7 Hz), 5.52 (2H, s), 6.90 (2H, d, J=8 Hz), 7.03 (2H, d, J=8 Hz), 7.37 (1H, d, J=8 Hz), 7.45 (1H, t, J=8 Hz), 7.55 (1H, t, J=8 Hz), 7.72 (1H, d, J=8 Hz), 8.54 (2H, s).

### Example 9

### Production of 1-[{2′-(1H-tetrazol-5-yl)biphenyl-4-yl}methyl]-2-butyl-4-(5-ethoxypyrimidin-2-yl)-1H-imidazole-5-carboxylic acid

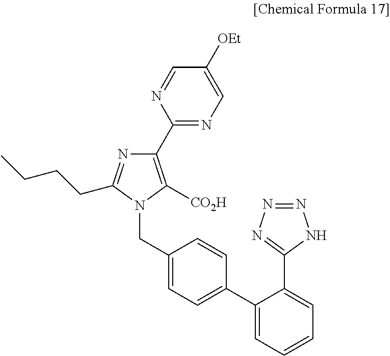

The target compound was obtained as a pale yellow amorphous (29.6%) according to the same reaction and treatment as the Process 1 of the Example 3 by using the methyl 1-[{2′-(1H-tetrazol-5-yl)biphenyl-4-yl}methyl]-2-butyl-4-(5-ethoxypyrimidin-2-yl)-1H-imidazole-5-carboxylic acid obtained in the Example 8 instead of the methyl 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid.

¹H-NMR (CD₃OD) δ: 0.89 (3H, t, J=7 Hz), 1.31-1.44 (5H, m), 1.56-1.64 (2H, m), 2.73 (2H, t, J=8 Hz), 4.48 (2H, q, J=7 Hz), 5.74 (2H, s), 7.04-7.12 (4H, m), 7.54 (2H, t, J=8 Hz), 7.63-7.67 (2H, m), 8.80 (2H, s).

### Example 10

### Production of 2-butyl-4-(4-fluorophenyl)-1-({2′-(5-oxo-4,5-dihydro-1,2,4-oxadiazol-3-yl)biphenyl-4-yl}methyl)-1H-imidazole-5-carboxamide

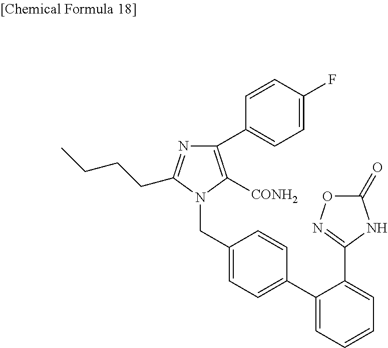

Process 1: 1-ethyl-3-(3-dimethylaminopropyl)carbodiimide hydrochloride (102 mg, 0.53 mmol) and 1-hydroxy benzotriazole (102 mg, 0.67 mmol) were added to the dichloromethane (2 mL) solution of the 2-butyl-1-{(2′-cyanobiphenyl-4-yl)methyl}-4-(4-fluorophenyl)-1H-imidazole-5-carboxylic acid (224 mg, 0.44 mmol) obtained in the Process 1 of the Example 2. The mixture was further added ammonia water (0.5 mL) and stirred at room temperature for 18 hours. The reaction mixture was diluted with chloroform, dried over anhydrous sodium sulfate, and concentrated in vacuo. The residues obtained were subjected to silica gel column chromatography (chloroform/methanol=20:1) to obtain 2-butyl-1-{(2′-cyanobiphenyl-4-yl)methyl}-4-(4-fluorophenyl)-1H-imidazole-5-carboxamide as a pale yellow solid (201 mg, quant.).

¹H-NMR (CDCl₃) δ: 0.91 (3H, t, J=7 Hz), 1.28-1.53 (2H, m), 1.65-1.80 (2H, m), 2.72 (2H, t, J=8 Hz), 5.67 (2H, s), 7.09-7.22 (4H, m), 7.41-7.57 (4H, m), 7.60-7.68 (3H, m), 7.76 (1H, dd, J=8, 1 Hz).

Process 2: Dimethylsulfoxide (2 mL) and sodium hydrogen carbonate (198 mg, 2.4 mmol) were added to hydroxylamine hydrochloride (139 mg, 2.0 mmol) and stirred at 40° C. for 1 hour. Dimethylsulfoxide (2 mL) solution of butyl-1-{(2′-cyanobiphenyl-4-yl)methyl}-4-(4-fluorophenyl)-1H-imidazole-5-carboxamide (53 mg, 0.118 mmol) was added to the reaction mixture and stirred at 90° C. for 24 hours. After completion of the reaction, the reaction mixture was added water and extracted with ethyl acetate. The organic layer was combined, washed with water and brine, dried over anhydrous sodium sulfate, and concentrated in vacuo. The residues obtained were subjected to silica gel column chromatography (chloroform/methanol=20:1) to obtain 4′-[{2-butyl-4-(4-fluorophenyl)-5-(carboxamide)-1H-imidazol-1-yl}methyl]-N′-hydroxybiphenyl-2-carboimidamide as a crude product (51 mg). 1,1-carbonyldiimidazole (57 mg, 0.35 mmol) and 1,8-diazabicyclo[5.4.0]undec-7-ene (54 mg, 0.35 mmol) were added to the N,N-dimethylformamide (5 mL) solution of 4′-[{2-butyl-4-(4-fluorophenyl)-5-(carboxamide)-1H-imidazol-1-yl}methyl]-N′-hydroxybiphenyl-2-carboimidamide obtained and stirred at room temperature for 1 hour. After completion of the reaction, the reaction mixture was added water and extracted with ethyl acetate. The organic layer was combined, washed with water and brine, dried over anhydrous sodium sulfate, and concentrated in vacuo. The residues obtained were purified and isolated by silica gel column chromatography (chloroform/methanol=20:1) to obtain the target compound as a white amorphous (23 mg, 37%).

¹H-NMR (CDCl₃) δ: 0.93 (3H, t, J=7 Hz), 1.38-1.51 (2H, m), 1.70-1.83 (2H, m), 2.75-2.85 (2H, m), 5.53 (2H, s), 5.81 (1H, br s), 7.04 (2H, d, J=9 Hz), 7.11 (2H, t, J=9 Hz), 7.29 (2H, d, J=8 Hz), 7.44-7.55 (2H, m), 7.58-7.67 (3H, m), 7.80 (1H, dd, J=8, 1 Hz).

### Example 11

### Production of 2-butyl-N-ethyl-4-(4-fluorophenyl)-1-[{2′-(5-oxo-4,5-dihydro-1,2,4-oxadiazol-3-yl)biphenyl-4-yl}methyl]-1H-imidazole-5-carboxamide

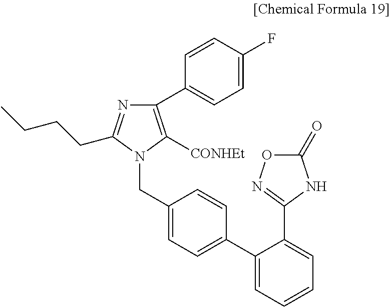

Process 1: 2-butyl-1-{(2′-cyanobiphenyl-4-yl)methyl}-N-ethyl-4-(4-fluorophenyl)-1H-imidazole-5-carboxamide was obtained as a white solid (99%) according to the same reaction and treatment as the Process 1 of the Example 10 by using ethylamine (12 mol/L aqueous solution) instead of the ammonia water.

¹H-NMR (CDCl₃) δ: 0.90 (3H, t, J=7 Hz), 0.94 (3H, t, J=7 Hz), 1.34-1.46 (2H, m), 1.68-1.78 (2H, m), 2.68-2.77 (2H, m), 3.17-3.29 (2H, m), 5.57 (2H, s), 7.10 (2H, t, J=9 Hz), 7.19 (2H, d, J=8 Hz), 7.40-7.54 (4H, m), 7.57-7.68 (3H, m), 7.75 (1H, dd, J=8, 1 Hz).

Process 2: The target compound was obtained as a white amorphous (47%) according to the same reaction and treatment as the Process 2 of the Example 10 by using 2-butyl-1-{(2′-cyanobiphenyl-4-yl)methyl}-N-ethyl-4-(4-fluorophenyl)-1H-imidazole-5-carboxamide instead of the 2-butyl-1-{(2′-cyanobiphenyl-4-yl)methyl}-4-(4-fluorophenyl)-1H-imidazole-5-carboxamide.

¹H-NMR (CDCl₃) δ: 0.92 (6H, t, J=7 Hz), 0.93 (6H, t, J=7 Hz), 1.36-1.49 (2H, m), 1.68-1.81 (2H, m), 2.63-2.73 (2H, m), 3.12-3.25 (2H, m), 5.52 (2H, s), 5.63 (1H, br s), 7.04-7.15 (4H, m), 7.31 (2H, d, J=9 Hz), 7.37-7.66 (6H, m), 7.80 (1H, dd, J=8, 1 Hz).

### Example 12

### Production of 2-butyl-N,N-diethyl-4-(4-fluorophenyl)-1-[{2′-(5-oxo-4,5-dihydro-1,2,4-oxadiazol-3-yl)biphenyl)-4-yl}methyl]-1H-imidazole-5-carboxamide

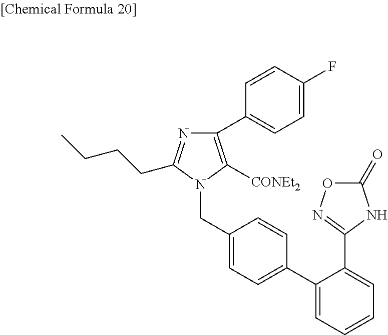

Process 1: 1-ethyl-3-(3-dimethylaminopropyl)carbodiimide hydrochloride (51 mg, 0.27 mmol) and 1-hydroxy benzotriazole (51 mg, 0.34 mmol) were added to the dichloromethane (1 mL) solution of 2-butyl-1-{(2′-cyanobiphenyl-4-yl)methyl}-4-(4-fluorophenyl)-1H-imidazole-5-carboxylic acid (112 mg, 0.22 mmol) obtained in the Process 1 of the Example 2. The mixture was further added diethyl amine (81 mg, 1.11 mmol) and stirred at room temperature for 18 hours. The reaction mixture was diluted with chloroform, dried over anhydrous sodium sulfate, and concentrated in vacuo. The residues obtained were subjected to silica gel column chromatography (chloroform/methanol=20:1) to obtain 2-butyl-1-{(2′-cyanobiphenyl-4-yl)methyl}-N,N-diethyl-4-(4-fluorophenyl)-1H-imidazole-5-carboxamide as a yellow oil (95 mg, 84%).

¹H-NMR (CDCl₃) δ: 0.35 (3H, t, J=7 Hz), 0.96 (3H, t, J=7 Hz), 1.02 (3H, t, J=7 Hz), 1.43-1.54 (2H, m), 1.72-1.89 (2H, m), 2.66-2.90 (4H, m), 3.08-3.58 (2H, m), 5.12-5.46 (2H, m), 7.03 (2H, t, J=9 Hz), 7.22-7.26 (2H, m), 7.40-7.68 (8H, m), 7.73-7.78 (1H, m).

Process 2: The target compound was obtained as a colorless and transparent oil (32%) according to the same reaction and treatment as the Process 2 of the Example 10 by using 2-butyl-1-{(2′-cyanobiphenyl-4-yl)methyl}-N,N-diethyl-4-(4-fluorophenyl)-1H-imidazole-5-carboxamide instead of the 2-butyl-1-{(2′-cyanobiphenyl-4-yl)methyl}-4-(4-fluorophenyl)-1H-imidazole-5-carboxamide.

¹H-NMR (CDCl₃) δ: 0.45 (3H, t, J=7 Hz), 0.94 (3H, t, J=7 Hz), 1.02 (3H, t, J=7 Hz), 1.34-1.51 (3H, m), 1.64-1.81 (2H, m), 2.58-2.82 (4H, m), 3.06-3.59 (2H, m), 5.06-5.38 (2H, m), 6.99 (2H, t, J=9 Hz), 7.16 (2H, d, J=8 Hz), 7.28-7.34 (2H, m), 7.43-7.52 (3H, m), 7.58 (1H, td, J=8, 1 Hz), 7.70 (1H, dd, J=8, 1 Hz).

### Example 13

### Production of 3-[4′-[{2-butyl-4-(4-fluorophenyl)-5-(morpholin-4-carbonyl)-1H-imidazol-1-yl}methyl]biphenyl-2-yl]-1,2,4-oxadiazol-5(4H)-one

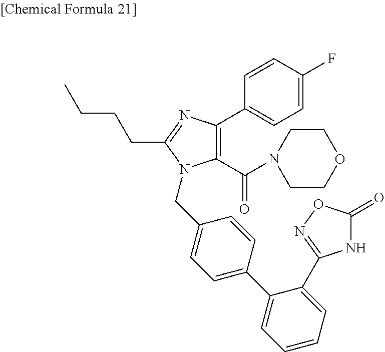

Process 1; 4′-[{2-butyl-4-(4-fluorophenyl)-5-(morpholin-4-carbonyl)-1H-imidazol-1-yl}methyl]biphenyl-2-carbonitrile was obtained as a white amorphous (86%) according to the same reaction and treatment as the Process 1 of the Example 2 by using morpholine instead of the diethyl amine.

¹H-NMR (CDCl₃) δ: 0.97 (3H, t, J=7 Hz), 1.40-1.55 (2H, m), 1.75-1.91 (2H, m), 2.59-2.80 (4H, m), 2.80-2.91 (2H, m), 3.28-3.69 (4H, m), 5.14-5.51 (OH, m), 7.06 (2H, t, J=9 Hz), 7.22 (2H, d, J=8 Hz), 7.42-7.57 (6H, m), 7.65 (1H, td, J=8, 1 Hz), 7.75 (1H, d, J=8 Hz).

Process 2: The target compound was obtained as a colorless and transparent oil (49%) according to the same reaction and treatment as the Process 2 of the Example 10 by using 4′-[{2-butyl-4-(4-fluorophenyl)-5-(morpholin-4-carbonyl)-1H-imidazol-1-yl}methyl]biphenyl-2-carbonitrile instead of the 2-butyl-1-{(2′-cyanobiphenyl-4-yl)methyl}-4-(4-fluorophenyl)-1H-imidazole-5-carboxamide.

¹H-NMR (CDCl₃) δ: 0.96 (38, t, J=7 Hz), 1.40-1.52 (2H, m), 1.72-1.87 (2H, m), 2.69-2.84 (6H, m), 3.30-3.67 (4H, m), 5.11-5.47 (2H, m), 7.05 (2H, t, J=9 Hz), 7.18 (2H, d, J=8 Hz), 7.29-7.36 (3H, m), 7.47-7.55 (3H, m), 7.57-7.66 (1H, m), 7.77 (1H, dd, J=8, 1 Hz).

### Example 14

### Production of 3-[4′-[{2-butyl-4-(4-fluorophenyl)-5-(pyrrolidin-1-carbonyl)-1H-imidazol-1-yl}methyl]biphenyl-2-yl]-1,2,4-oxadiazol-5(4H)-one

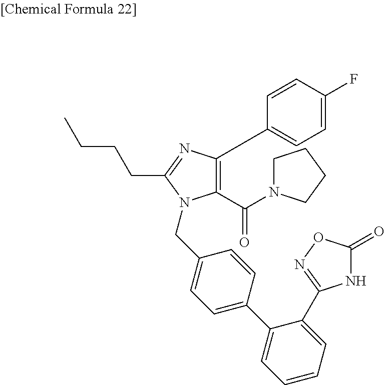

Process 1: 4′-[{2-butyl-4-(4-fluorophenyl)-5-(pyrrolidin-1-carbonyl)-1 H-imidazol-1-yl}methyl]biphenyl-2-carbonitrile was obtained as a yellow oil (98%) according to the same reaction and treatment as the Process 1 of the Example 12 by using pyrrolidine instead of the diethyl amine.

¹H-NMR (CDCl₃) δ: 0.97 (3H, t, J=7 Hz), 1.23-1.33 (2H, m), 1.42-1.58 (4H, m), 1.74-1.90 (2H, m), 2.55 (2H, t, J=7 Hz), 2.84 (2H, t, J=8 Hz), 3.35 (2H, t, J=7 Hz), 5.31 (2H, s), 7.03 (2H, t, J=9 Hz), 7.20-7.26 (2H, m), 7.41-7.69 (7H, m), 7.76 (1H, dd, J=8, 1 Hz).

Process 2: The target compound was obtained as a colorless and transparent oil (37%) according to the same reaction and treatment as the Process 2 of the Example 10 by using 4′-[{2-butyl-4-(4-fluorophenyl)-5-(pyrrolidin-1-carbonyl)-1 H-imidazol-1-yl}methyl]biphenyl-2-carbonitrile instead of the 2-butyl-1-{(2′-cyanobiphenyl-4-yl)methyl}-4-(4-fluorophenyl)-1H-imidazole-5-carboxamide.

¹H-NMR (CDCl₃) δ: 0.95 (3H, t, J=7 Hz), 1.32-1.52 (6H, m), 1.69-1.84 (2H, m), 2.64 (2H, t, J=7 Hz), 2.74 (2H, t, J=8 Hz), 3.37 (2H, t, J=7 Hz), 5.26 (2H, s), 7.01 (2H, t, J=9 Hz), 7.16 (2H, d, J=8 Hz), 7.32 (2H, d, J=9 Hz), 7.45-7.64 (5H, m), 7.77 (1H, d, J=8 Hz).

### Example 15

### Production of 1-[{2′-(1H-tetrazol-5-yl)biphenyl-4-yl}methyl]-2-butyl-4-(4-fluorophenyl)-1H-imidazole-5-carboxamide

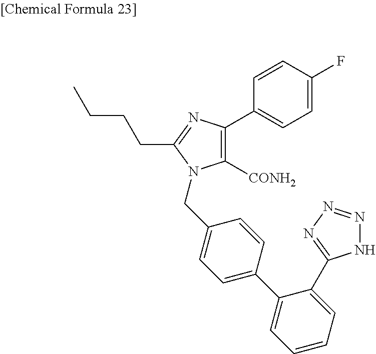

The target compound was obtained as a white amorphous (47%) according to the same reaction and treatment as the Process 1 of the Example 5 by using the 2-butyl-1-{(2′-cyanobiphenyl-4-yl)methyl}-4-(4-fluorophenyl)-1H-imidazole-5-carboxamide obtained in the Process 1 of the Example 10 instead of the 4′-[{2-butyl-4-(5-ethoxypyrimidin-2-yl)-5-(hydroxymethyl)-1 H-imidazol-1-yl}methyl]biphenyl-2-carbonitrile.

¹H-NMR (CDCl₃) δ: 0.88 (3H, t, J=7 Hz), 1.23-1.43 (2H, m), 1.56-1.73 (2H, m), 2.56 (2H, t, J=8 Hz), 5.47 (2H, s), 5.56-5.89 (2H, m), 6.86 (2H, d, J=8 Hz), 6.97-7.08 (4H, m), 7.35-7.62 (5H, m), 7.84 (1H, d, J=7 Hz).

### Example 16

### Production of 1-[{2′-(1H-tetrazol-5-yl)biphenyl-4-yl}methyl]-2-butyl-N-ethyl-4-(4-fluorophenyl)-1H-imidazole-5-carboxamide

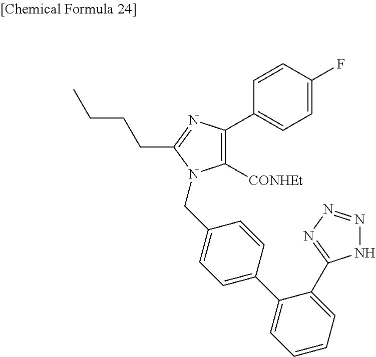

The target compound was obtained as a white amorphous (71%) according to the same reaction and treatment as the Process 1 of the Example 5 by using the 2-butyl-1-{(2′-cyanobiphenyl-4-yl)methyl}-N-ethyl-4-(4-fluorophenyl)-1H-imidazole-5-carboxamide obtained in the Process 1 of the Example 11 instead of the 4′-[{2-butyl-4-(5-ethoxypyrimidin-2-yl)-5-(hydroxymethyl)-1H-imidazol-1-yl}methyl]biphenyl-2-carbonitrile.

¹H-NMR (CDCl₃) δ: 0.88 (3H, t, J=7.3 Hz), 0.89 (3H, t, J=7.3 Hz), 1.25-1.41 (3H, m), 1.57-1.69 (2H, m), 2.40 (2H, t, J=7.9 Hz), 3.09-3.19 (2H, m), 5.44 (2H, s), 5.53 (1H, t, J=5.4 Hz), 6.93 (4H, dd, J=14.2, 8.2 Hz), 7.07 (2H, d, J=7.9 Hz), 7.25-7.33 (2H, m), 7.38 (1H, d, J=7.6 Hz), 7.44-7.61 (2H, m), 7.76 (1H, d, J=7.3 Hz).

### Example 17

### Production of 1-[{2′-(1H-tetrazol-5-yl)biphenyl-4-yl}methyl]-2-butyl-N,N-diethyl-4-(4-fluorophenyl)-1H-imidazole-5-carboxamide

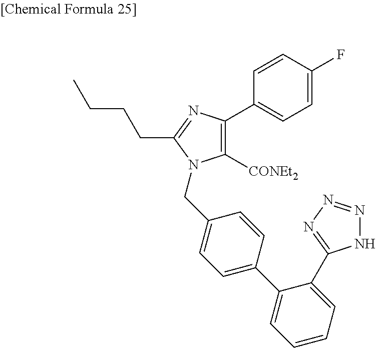

The target compound was obtained as a colorless and transparent oil (38%) according to the same reaction and treatment as the Process 1 of the Example 5 by using the 2-butyl-1-{(2′-cyanobiphenyl-4-yl)methyl}-N,N-diethyl-4-(4-fluorophenyl)-1H-imidazole-5-carboxamide obtained in the Process 1 of the Example 12 instead of the 4′-[{2-butyl-4-(5-ethoxypyrimidin-2-yl)-5-(hydroxymethyl)-1H-imidazol-1-yl}methyl]biphenyl-2-carbonitrile.

¹H-NMR (CDCl₃) δ: 0.44 (3H, t, J=7 Hz), 0.90 (3H, t, J=7 Hz), 1.00 (3H, t, J=7 Hz), 1.37 (2H, td, J=15, 7 Hz), 1.56-1.75 (2H, m), 2.52-2.86 (4H, m), 3.11-3.55 (2H, m), 4.93-5.28 (2H, m), 6.90 (2H, t, J=9 Hz), 7.00 (2H, d, J=8 Hz), 7.07 (2H, d, J=8 Hz), 7.29-7.37 (3H, m), 7.45 (1H, td, J=8, 1 Hz), 7.54 (1H, td, J=8, 1 Hz), 7.75 (1H, dd, J=8, 1 Hz).

### Example 18

### Production of [1-[{2′-(1H-tetrazol-5-yl)biphenyl-4-yl}methyl]-2-butyl-4-(4-fluorophenyl)-1H-imidazol-5-yl](morpholino)methanone

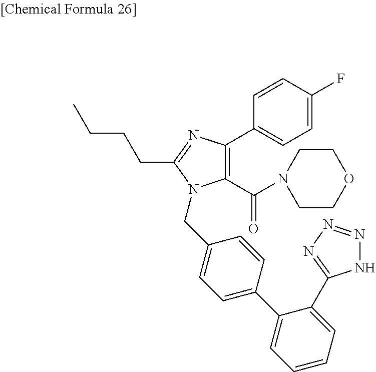

The target compound was obtained as a colorless and transparent oil (36%) according to the same reaction and treatment as the Process 1 of the Example 5 by using the 4′-[{2-butyl-4-(4-fluorophenyl)-5-(morpholin-4-carbonyl)-1H-imidazol-1-yl}methyl]biphenyl-2-carbonitrile obtained in the Process 1 of the Example 13 instead of the 4′-[{2-butyl-4-(5-ethoxypyrimidin-2-yl)-5-(hydroxymethyl)-1H-imidazol-1-yl}methyl]biphenyl-2-carbonitrile.

¹H-NMR (CDCl₃) δ: 0.94 (3H, t, J=7 Hz), 1.43 (2H, td, J=15, 7 Hz), 1.68-1.79 (2H, m), 2.52-2.98 (6H, m), 3.19-3.72 (4H, m), 5.02-5.35 (2H, m), 6.95-7.03 (4H, m), 7.11 (2H, d, J=8 Hz), 7.32 (1H, dd, J=8, 1 Hz), 7.40-7.45 (2H, m), 7.49 (1H, td, J=8, 1 Hz), 7.56 (1H, td, J=8, 1 Hz), 7.87 (18, dd, J=8, 1 Hz).

### Example 19

### Production of [1-[{2′-(1H-tetrazol-5-yl)biphenyl-4-yl}methyl]-2-butyl-4-(4-fluorophenyl)-1H-imidazol-5-yl](pyrrolidin-1-yl)methanone

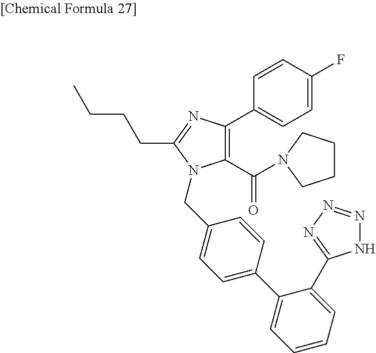

The target compound was obtained as a colorless and transparent oil (36%) according to the same reaction and treatment as the Process 1 of the Example 5 by using the 4′-[{2-butyl-4-(4-fluorophenyl)-5-(pyrrolidin-1-carbonyl)-1H-imidazol-1-yl}methyl]biphenyl-2-carbonitrile obtained in the Process 1 of the Example 14 instead of the 4′-[{2-butyl-4-(5-ethoxypyrimidin-2-yl)-5-(hydroxymethyl)-1 H-imidazol-1-yl}methyl]biphenyl-2-carbonitrile.

¹H-NMR (CDCl₃) δ: 0.91 (3H, t, J=7 Hz), 1.32-1.45 (4H, m), 1.60-1.72 (4H, m), 2.52-2.64 (4H, m), 3.34 (2H, t, J=7 Hz), 5.14 (2H, s), 6.92 (2H, t, J=9 Hz), 6.96 (2H, d, J=8 Hz), 7.05 (2H, d, J=8 Hz), 7.29-7.39 (3H, m), 7.47 (1H, t, J=8 Hz), 7.55 (1H, t, J=8 Hz), 7.77 (1H, d, J=8 Hz).

### Test Example 1

### Angiotensin II Antagonistic Activity in Isolated Rabbit Blood Vessels

By using a specimen of isolated rabbit blood vessels, antagonistic activity of the compounds of the invention against angiotensin II type 1 receptor was estimated from a dose-response curve of angiotensin II-induced blood vessel contraction. Specifically, the specimen of thoracic aorta ring of a rabbit (New Zealand White: male, 2.4 to 3.0 kg) was suspended in a magnus bath filled with Krebs-Henseleite buffer (composition: 118 mM NaCl, 4.7 mM KCl, 2.55 mM CaCl₂, 1.18 mM MgSO₄, 1.18 mM KH₂PO₄, 24.88 mM NaHCO₃, and 11.1 mM D-glucose), and angiotensin II (10 nM)-induced contraction was obtained in the presence of the compounds of each Example (1 nmol/L to 10 μmol/L). During the measurement, the inside temperature of the magnus bath was maintained at 37° C. and the bath was continuously ventilated with a sufficient amount of mixed gas (95% O₂ and 5% CO₂). The angiotensin II-induced contraction was converted into a relative value (%) that is based on the angiotensin II (10 nM)-induced contraction in the absence of the compounds of each Example. From the concentration-response curve obtained therefrom, 50% inhibition concentration (IC₅₀ value) was calculated by using SAS Preclinical Package Ver 5.0 (trade name, manufactured by SAS institute Japan Co., Tokyo, Japan), which is a statistical analysis program. The activity value of the compounds is given in the Table 1. As described in the Table 1, it was confirmed that the compounds of the invention have a potent angiotensin II antagonistic activity.

TABLE 1

| | Angiotensin II 50% |
|---|---|
| Example No. | inhibition concentration (μM) |
| 2 | 4.07 |
| 4 | 1.04 |
| 5 | 0.77 |
| 6 | 6.94 |
| 8 | 1.01 |
| 10 | 0.80 |
| 11 | 5.69 |
| 15 | 0.46 |
| 16 | 0.99 |
| 18 | 1.60 |
| 19 | 6.98 |

### Test Example 2

### PPARγ Activation Activity

The agonistic activity of the compounds of the invention on PPARγ was measured based on the transfection assay using COS7 cells (DS Pharma Biomedical Co., Ltd., Osaka, Japan), which are the cell line derived from the kidney of the African green monkey. COS7 cells were cultured under 5% CO₂ concentration, and DMEM medium containing 10% fetal bovine serum, glutamic acid, and antibiotics was used as a medium.

As an expression vector, a chimera in which DNA binding domain of Gal4, which is a yeast transcription factor, and ligand binding domain of human PPARγ2 are fused, i.e., a fused product between the amino acids 1 to 147 of Gal4 transcription factor and the amino acids 182 to 505 of human PPARγ2, was used. Furthermore, as a reporter vector, a firefly luciferase containing five copies of Gal4 recognition sequence in the promoter region was used. Plasmid transfection to the cells was performed according to a method which uses jetPEI (trade name, manufactured by Funakoshi Co., Ltd., Tokyo, Japan). Furthermore, β-galactosidase expression vector was employed as an internal standard.

After the transfection of the cells, the medium was replaced with a DMEM medium (containing 1 serum) added with the test compound, and the cells were further cultured for 16 hours. After that, the luciferase activity and β-galactosidase activity in the cell lysis solution were measured.

For the present test, dimethyl sulfoxide (DMSO) was used for dissolution and dilution of the test compounds, and during the cell treatment, the DMSO concentration in DMEM medium (containing 1% serum) was adjusted to 0.1%. The 50% effective concentration of the test compound (EC₅₀, 50% effect concentration) was calculated by using SAS Preclinical Package Ver 5.0 (trade name, manufactured by SAS institute Japan Co., Tokyo, Japan), which is a statistical analysis program.

The results are given in the Table 2. As described in the Table 2, it was confirmed that the compounds of the invention have a potent PPARγ, activation activity. Under the same condition, the PPARγ activation activity of telmisartan, that is, EC₅₀, was 1 μM to 5 μM. Further, it was also confirmed that the compounds of the Examples that are not described in the Table 2 also have the PPARγ activation activity at the concentration of 30 μM.

TABLE 2

| Example No. | EC₅₀ (μM) |
|---|---|
| 1 | 1.52 |
| 4 | 1.48 |
| 6 | 0.56 |
| 8 | 3.01 |
| 10 | 3.43 |
| 11 | 2.28 |
| 12 | 2.37 |
| 17 | 3.30 |
| 18 | 4.32 |

From the results obtained above, it was confirmed that the compounds represented by the general formula (I) have both a potent angiotensin II receptor antagonistic activity and a PPARγ activation activity. Thus, it was found that the compounds (I) of the invention and pharmaceutically acceptable salts thereof are useful as an effective component of a prophylactic and/or therapeutic agent for disorders involved with angiotensin II and PPARγ, for example, hypertension, heart diseases, angina pectoris, cerebrovascular disorders, cerebral circulatory disorders, ischemic peripheral circulatory disorders, renal diseases, arteriosclerosis, inflammatory diseases, type 2 diabetes, diabetic complications, insulin resistance syndrome, syndrome X, metabolic syndrome, and hyperinsulinemia.

### INDUSTRIAL APPLICABILITY

The invention provides a novel compound of 1-(biphenyl-4-yl-methyl)-1H-imidazole derivative represented by the formula (I) or a salt thereof, or a solvate thereof, which has both an angiotensin II receptor antagonistic activity and a PPARγ activation activity. The compound can be used as an effective component of a novel pharmaceutical product which is useful as a prophylactic and/or therapeutic agent for disorders that are related with angiotensin II and PPARγ, for example, hypertension, heart diseases, angina pectoris, cerebrovascular disorders, cerebral circulatory disorders, ischemic peripheral circulatory disorders, renal diseases, arteriosclerosis, inflammatory diseases, type 2 diabetes, diabetic complications, insulin resistance syndrome, syndrome X, metabolic syndrome, and hyperinsulinemia, and therefore have an industrial applicability.

1. A 1-(biphenyl-4-yl-methyl)-1H-imidazole derivative represented by the formula (I) below or a salt thereof, or a solvate thereof: 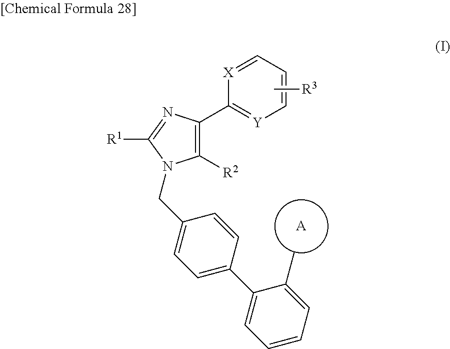
[in the formula, ring A represents the following formula (II) or the following formula (III): 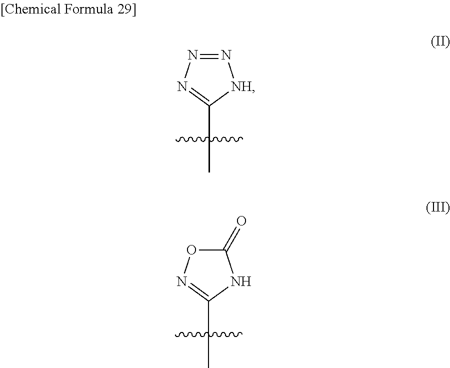
R¹ represents a C₁₋₆ alkyl group,
R² represents a C₁₋₆ alkyl group which may be substituted with a hydroxy group, or —CO—R⁴ (wherein, R⁴ represents a hydroxy group, a C₁₋₆ alkoxy group, an amino group, a mono(C₁₋₆ alkyl)amino group, a di(C₁₋₆ alkyl)amino group, a morpholino group, a piperidino group, or a pyrrolidino group),
R³ represents a halogen atom or a C₁₋₆ alkoxy group, and
X and Y, which are the same or different each other, represent a nitrogen atom or CH].

2. The 1-(biphenyl-4-yl-methyl)-1H imidazole derivative described in claim 1, or salt thereof, or solvate thereof, in which the compound represented by the formula (I) is at least one compound selected from the group consisting of:
3-[4′-[{2-butyl-4-(4-fluorophenyl)-5-(hydroxymethyl)-1H-imidazol-1-yl}methyl]biphenyl-2-yl]-1,2,4-oxazole-5(4H)-one,
methyl 2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid,
2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxylic acid,
3-[4′-[{2-butyl-4-(5-ethoxypyrimidin-2-yl)-5-(hydroxymethyl)-1H-imidazol-1-yl}methyl]-biphenyl-2-yl]-1,2,4-oxadiazol-5(4H)-one,
[1-[{2′-(1H-tetrazol-5-yl)biphenyl-4-yl}methyl]-2-butyl-4-(5-ethoxypyrimidin-2-yl)-1H-imidazol-5-yl]methanol,
methyl 2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid,
2-butyl-4-(5-ethoxypyrimidin-2-yl)-1-[{2′-(5-oxo-4,5-dihydro-1,2,4-oxadiazol-3-yl)biphenyl-4-yl}methyl]-1H-imidazole-5-carboxylic acid,
methyl 1-[{2′-(1H-tetrazol-5-yl)biphenyl-4-yl}methyl]-2-butyl-4-(5-ethoxypyrimidin-2-yl)-1H-imidazole-5-carboxylic acid,
1-[{2′-(1H-tetrazol-5-yl)biphenyl-4-yl}methyl]-2-butyl-4-(5-ethoxypyrimidin-2-yl)-1H-imidazole-5-carboxylic acid,
2-butyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
2-butyl-N-ethyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
2-butyl-N,N-diethyl-4-(4-fluorophenyl)-1-[{2′-(5-oxo-4,5-dihydro-1,2,4-oxadiazol-3-yl)biphenyl-4-yl}methyl]-1H-imidazole-5-carboxamide,
3-[4′-[{2-butyl-4-(4-fluorophenyl)-5-(morpholin-4-carbonyl)-1H-imidazol-1-yl}methyl]biphenyl-2-yl]-1,2,4-oxadiazol-5(4H)-one,
3-[4′-[{2-butyl-4-(4-fluorophenyl)-5-(pyrrolidin-1-carbonyl)-1H-imidazol-1-yl}methyl]biphenyl-2-yl]-1,2,4-oxadiazol-5(4H)-one,
1-[{2′-(1H-tetrazol-5-yl)biphenyl-4-yl}methyl]-2-butyl-4-(4-fluorophenyl)-1H-imidazole-5-carboxamide,
1-[{2′-(1H-tetrazol-5-yl)biphenyl-4-yl}methyl]-2-butyl-N-ethyl-4-(4-fluorophenyl)-1H-imidazole-5-carboxamide,
1-[{2′41H-tetrazol-5-yl)biphenyl-4-yl}methyl]-2-butyl-N,N-diethyl-4-(4-fluorophenyl)-1H-imidazole-5-carboxamide,
[1-[{2′-(1H-tetrazol-5-yl)biphenyl-4-yl}methyl]-2-butyl-4-(4-fluorophenyl)-1H-imidazol-5-yl](morpholino)methanone, and
[1-[{2′-(1H-tetrazol-5-yl)biphenyl-4-yl}methyl]-2-butyl-4-(4-fluorophenyl)-1H-imidazol-5-yl](pyrrolidin-1-yl)methanone.

3. A pharmaceutical composition comprising the 1-(biphenyl-4-yl-methyl)-1H-imidazole derivative or salt thereof, or solvate thereof according to claim 1 and a pharmaceutically acceptable carrier.

4. A pharmaceutical composition having both angiotensin II receptor antagonistic activity and PPARγ activation activity in which the 1-(biphenyl-4-yl-methyl)-1H-imidazole derivative or salt thereof, or solvate thereof according to claim 1 is comprised as an effective component.

5. An agent for preventing and/or treating a circulatory disorder which comprises as an effective component the 1-(biphenyl-4-yl-methyl)-1H-imidazole derivative or salt thereof, or solvate thereof according to claim 1.

6. The agent for preventing and/or treating a circulatory disorder according to claim 5, wherein the circulatory disorder is hypertension, heart diseases, angina pectoris, cerebrovascular disorders, cerebral circulatory disorders, ischemic peripheral circulatory disorders, renal diseases, or arteriosclerosis.

7. An agent for preventing and/or treating a metabolic disorder comprising as an effective component the 1-(biphenyl-4-yl-methyl)-1H-imidazole derivative or salt thereof, or solvate thereof according to claim 1.

8. The agent for preventing and/or treating a metabolic disorder according to claim 7, wherein the metabolic disorder is type 2 diabetes, diabetic complications (diabetic retinopathy, diabetic neuropathy, or diabetic nephropathy), insulin resistance syndrome, metabolic syndrome, or hyperinsulinemia.

9. A method of preventing and/or treating a circulatory disorder, comprising administering an effective amount of the 1-(biphenyl-4-yl-methyl)-1H-imidazole derivative or salt thereof, or solvate thereof according to claim 1 to a patient who is in need of treatment.

10. A method of preventing and/or treating a metabolic disorder, comprising administering an effective amount of the 1-(biphenyl-4-yl-methyl)-1H-imidazole derivative or salt thereof, or solvate thereof according to claim 1 to a patient who is in need of treatment.

11. Use of the 1-(biphenyl-4-yl-methyl)-1H-imidazole derivative or salt thereof, or solvate thereof according to claim 1 for producing a preparation for preventing and/or treating a circulatory disorder.

12. Use of the 1-(biphenyl-4-yl-methyl)-1H-imidazole derivative or salt thereof, or solvate thereof according to claim 1 for producing a preparation for preventing and/or treating a metabolic disorder.

13. The 1-(biphenyl-4-yl-methyl)-1H-imidazole derivative or salt thereof, or solvate thereof according to claim 1 as an agent for prevention and/or treatment having both angiotensin II receptor antagonistic activity and PPARγ activation activity.

14. A pharmaceutical composition comprising the 1-(biphenyl-4-yl-methyl)-1H-imidazole derivative or salt thereof, or solvate thereof according to claim 2 and a pharmaceutically acceptable carrier.

15. A pharmaceutical composition having both angiotensin II receptor antagonistic activity and PPARγ activation activity in which the 1-(biphenyl-4-yl-methyl)-1H-imidazole derivative or salt thereof, or solvate thereof according to claim 2 is comprised as an effective component.

16. An agent for preventing and/or treating a circulatory disorder which comprises as an effective component the 1-(biphenyl-4-yl-methyl)-1H-imidazole derivative or salt thereof, or solvate thereof according to claim 2.

17. An agent for preventing and/or treating a metabolic disorder comprising as an effective component the 1-(biphenyl-4-yl-methyl)-1H-imidazole derivative or salt thereof, or solvate thereof according to claim 2.

18. A method of preventing and/or treating a circulatory disorder, comprising administering an effective amount of the 1-(biphenyl-4-yl-methyl)-1H-imidazole derivative or salt thereof, or solvate thereof according to claim 2 to a patient who is in need of treatment.

19. A method of preventing and/or treating a metabolic disorder, comprising administering an effective amount of the 1-(biphenyl-4-yl-methyl)-1H-imidazole derivative or salt thereof, or solvate thereof according to claim 2 to a patient who is in need of treatment.

20. Use of the 1-(biphenyl-4-yl-methyl)-1H-imidazole derivative or salt thereof, or solvate thereof according to claim 2 for producing a preparation for preventing and/or treating a circulatory disorder.

21. Use of the 1-(biphenyl-4-yl-methyl)-1H-imidazole derivative or salt thereof, or solvate thereof according to claim 2 for producing a preparation for preventing and/or treating a metabolic disorder.

22. The 1-(biphenyl-4-yl-methyl)-1H-imidazole derivative or salt thereof, or solvate thereof according to claim 2 as an agent for prevention and/or treatment having both angiotensin II receptor antagonistic activity and PPARγ activation activity.
